(19) United States
(12) Patent Application Publication
(10) Pub. No.: US 2025/0239327 A1
(43) Pub. Date: Jul. 24, 2025

(54) LIQUID BIOPSY ASSAY FOR GENOMIC PROFILING OF CIRCULATING TUMOR DNA

(71) Applicant: BillionToOne, Inc., Menlo Park, CA (US)

(72) Inventors: Jan Christian WIGNALL, San Francisco, CA (US); Wen ZHOU, Sunnyvale, CA (US); Xavier Scott BOWER, Burlingame, CA (US); Patrick Daniel CHERRY, San Francisco, CA (US); Michael Quinlan O'SULLIVAN, Redwood City, CA (US); Zeqian LI, Menlo Park, CA (US); Oguzhan ATAY, Menlo Park, CA (US); David TSAO, San Carlos, CA (US)

(21) Appl. No.: 19/033,133

(22) Filed: Jan. 21, 2025

Related U.S. Application Data
(63) Continuation-in-part of application No. PCT/US25/12270, filed on Jan. 19, 2025.
(60) Provisional application No. 63/623,108, filed on Jan. 19, 2024.

(51) Int. Cl. G16B 20/20; C12N 15/10; C12Q 1/6806; C12Q 1/6855; C12Q 1/6869; G16B 20/10; G16B 40/10; G16B 40/30

(52) U.S. Cl. CPC G16B 20/20; C12N 15/1089; C12Q 1/6806; C12Q 1/6855; C12Q 1/6869; G16B 20/10; G16B 40/10; G16B 40/30; C12Q 2600/156

(57) ABSTRACT

An assay provides comprehensive genomic profiling for plasma-derived circulating tumor DNA from solid tumors. The assay covers genes for mutations including single nucleotide variants (SNVs), insertions and deletions (indels), copy number variants (CNVs), and fusions, as well as microsatellite instability (MSI) status. The assay may use a custom hybrid capture process with optimized chemistry and panel design, as well as novel algorithms for SNV, indel, and CNV data filtering to optimize performance.

### CROSS-REFERENCE TO RELATED APPLICATIONS

This application is a continuation-in-part of International Application No. PCT/US2025/012270, filed Jan. 19, 2025, which claims the benefit of U.S. Provisional Patent Application No. 63/623,108, filed Jan. 19, 2024, both of which are incorporated by reference.

### BACKGROUND

### 1. Technical Field

The subject matter described relates generally to clinical assays and, in particular, to a liquid biopsy assay for genomic profiling of circulating tumor DNA.

### 2. Background Information

Liquid biopsy assays have emerged as vital tools in precision oncology, improving treatment selection for cancer patients by non-invasively identifying somatic mutations in the tumor, many of which have corresponding targeted therapies. Among these assays, Comprehensive Genomic Profiling (CGP) tests have been particularly effective in improving treatment selection for late-stage solid tumor cancer patients by allowing for the detection via circulating tumor DNA (ctDNA) of somatic mutations in the tumor, many of which have corresponding targeted therapies. However, technical restrictions in the performance of such tests have limited their utility when ctDNA signal is low.

The presence of low allele fractions or low level of copy number change signal in plasma does not necessarily mean low amounts of the mutation in the tumor itself but may rather be a reflection of low ctDNA shedding into the bloodstream. Nonetheless, tumors with sensitizing mutations at a low Variant Allele Frequency (VAF) are no less susceptible to targeted therapies when properly identified. Recent studies in NSCLC have shown that as many as 25% of somatic mutations were found below 0.2% VAF (5), however the limit of detection of the currently available commercial assays are reported to be at 0.2% or greater. Therefore, there is an unmet clinical need to be able to identify somatic mutations below a 0.2% VAF to identify more patients who can benefit from targeted therapy. More broadly, there is an on-going need for more comprehensive and more accurate assays and data analysis techniques.

### SUMMARY

An assay can provide comprehensive genomic profiling test for plasma-derived circulating tumor DNA (ctDNA) from late-stage solid tumors. is the assay may be optimized for high diagnostic yield. In one embodiment, the assay covers 84 genes for mutations including single nucleotide variants (SNVs), insertions and deletions (indels), copy number variants (CNVs), and fusions, as well as microsatellite instability (MSI) status. The assay may utilize a custom hybrid capture process with optimized chemistry and panel design, as well as novel algorithms for SNV, indel, and CNV data filtering to optimize performance.

In addition, proprietary Quantitative Counting Template (QCT) technology allows for quality tracking across multiple processing steps, contributing to clinical utility due to more stringent quality control. These optimizations may provide assay sensitivity that surpasses that of other commercially available tests for multiple mutation types, detecting more pathogenic and actionable mutations at low tumor DNA fraction while maintaining similarly high specificity. In one analytical validation, the limit of detection for each mutation was determined to be 0.13-0.16% VAF for SNVs and indels, 2.10-2.16 copies for copy number amplifications, 1.7-1.9 copies for copy number losses, 0.25%-0.40% VAF for fusions, and 0.07%-0.40% tumor fraction for MSI. This superior analytical performance translates to clinical utility as well, with a study of clinical samples (N=481) from a variety of tumor types yielding only 9 (1.9%) samples with no mutations detected.

A head-to-head comparison with other commercial liquid biopsy assays on a subset of the clinical samples (N=182) showed that the assay detected 50.8% additional pathogenic small mutations and 109% additional CNVs, leading to a higher proportion of patients with at least one pathogenic mutation detected (89%) compared to comparators (80%). These results demonstrate that the assay is an innovative and high-performing liquid biopsy test, leveraging quantitative approaches in chemistry, bioinformatics, and process design to offer superior analytical and clinical performance compared to the current landscape of liquid biopsy assays.

The copy number signal from the assay may be used in a robust method of tumor fraction estimation and tissue copy number inference. Estimating tumor fraction and inferring copy number alterations (CNAs) or copy number variants (CNVs) from a cell-free DNA (cfDNA) liquid biopsy is challenging due to single nucleotide variant (SNV) heterogeneity and potential lack of SNV signal. About 90% of solid tumors exhibit chromosome arm level gains or losses (see Ben-David, U., Amon, A. Nat Rev Genet 21, 44-62 (2020); see also Sansregret, L., Bakhoum, S. F., et al. Genome Medicine, 13 (1), 93 (2021)), and this effect on copy number can be used as a tumor load biomarker. Aneuploidy signal in cfDNA can be used to estimate tumor fraction and infer tissue copy number, addressing a limitation of variant-based cfDNA assays and providing a robust method for benchmarking against gold-standard techniques like FISH.

In some embodiments, a method for genomic profiling of plasma-derived circulating tumor DNA (ctDNA) includes obtaining a mixture including plasma from a blood sample of a subject, the mixture including cell-free DNA (cfDNA) from the subject and performing processing steps on the mixture to produce a sequencing library. The sequencing library is sequenced to produce sequence reads. The method also includes constructing a tumor mutational profile for the subject based on the sequence reads. Constructing the tumor mutation profile includes performing copy number variant calling with normalization and auto-exclusion based on an aneuploidy signal. The tumor mutational profile may also include an indication of presence or absence of at least of single nucleotide variants (SNVs), insertions and deletions (indels), fusions, or microsatellite instability (MSI). The tumor mutational profile may include as indication of presence of a tumor mutation having a variant allelic fraction (VAF) in a range from 0.05% to 0.2%.

In some embodiments, the method also includes estimating tumor fraction from cell-free DNA (cfDNA). The tumor mutation profile can be further based on the estimated tumor fraction. 5. Estimating the tumor fraction can include applying a Gaussian Mixture Model (GMM) to quantify a liquid aneuploidy signal on measured genes and estimating the tumor fraction based on a periodicity pattern determined by the GMM.

In some embodiments, the method further includes inferring copy number alterations (CNAs) from cfDNA. The tumor mutation profile can be further based on the inferred copy number alterations. The method may use a panel of multiple genes with a limit of detection of 0.13-0.16% allele fraction for SNVs and indels, and 2.100-2.160 for Copy Number amplification, 1.7-1.9 copies for Copy Number loss, 0.25-0.40% allele fraction for fusions, and 0.07-0.40% allele fraction for MSI.

In some embodiments, at least some of the processing steps including adding a corresponding set of Quantitative Counting Template (QCT) molecules to the mixture. The method may also include performing quality tracking based on a subset of the sequence reads that correspond to the QCT molecules with the tumor mutation profile being constructed responsive to the quality tracking indicating success of the processing steps. The processing steps may include a cfDNA extraction step that extracts cfDNA from the plasma sample, during or before which extraction QCTs are added, wherein a number of sequence reads corresponding to the extraction QCTs indicates success or an issue with the cfDNA extraction step. Additionally or alternatively, the processing steps may include a library preparation step that includes at least one of end repair and adaptor ligation, during or before which library preparation QCTs are added, wherein a number of sequence reads corresponding to the library preparation QCTs indicates success of or an issue with the library preparation step. Additionally or alternatively, the processing steps comprise a selective enrichment step that selectively amplifies target sequences, during or before which target enrichment QCTs are added, wherein a number of sequence reads corresponding to the target enrichment QCTs indicates success of or an issue with the selective enrichment step. The sequencing may have a median probe coverage of at least 40,000×.

In some embodiments, the quality tracking includes calculating one or more QCT metrics from the sequence reads that correspond to the QCT molecules and comparing the one or more QCT metrics to one or more corresponding thresholds, wherein the success of the processing steps is indicated by the one or more QCT metrics meeting requirements relative to the corresponding thresholds. The one or more QCT metrics may include at least one of a z-score, a sequence saturation level, or a base-wise error rate.

In some embodiments, the method also includes false-positive filtering and calling of SNVs and Indels. The false-positive filtering may be adaptive filtering.

In some embodiments, the method further includes performing copy number noise reduction for a batch comprising a plurality of samples, the plurality of samples including the blood sample. Performing the copy number noise reduction may include: averaging raw coverages per gene per sample of the plurality of samples, the raw coverages measured at a center of each of a plurality of probes; normalizing the normalized coverages to a median gene-level coverage; filtering the normalized coverages for well-behaved probes; creating a model for each sample to predict sample-normalized coverage for guanine-cytosine (GC) content of probes; normalizing the sample-normalized coverages to expected values from the models corresponding to the samples to determine per-sample GC normalized coverage; removing, for all of the plurality of samples, probe values that deviate from the expected value by at least a threshold amount for at least one of the plurality of samples; measuring a median per-sample GC normalized coverage for each probe across the batch; normalizing the per-sample GC normalized coverage to generate expected GC normalized values; and calculating probe copy numbers from the expected GC normalized values.

In some embodiments, the method also includes estimating an in-tissue copy number of a focal CNV from a plasma measurement. The method can also include extracting buffy coat DNA from the blood sample; analyzing the buffy coat DNA to identify a set of one or more mutations present in the buffy coat DNA; and filtering mutations included in the tumor mutation profile to not include at least some of the set of one or more mutations. Analyzing the buffy coat DNA can include performing a Clonal Hematopoiesis of Indeterminate Potential (CHIP) analysis, using digital droplet PCR (ddPCR), performing Comprehensive Genome Profiling to identify CHIP mutations, or performing multiplex PCR to identify CHIP mutations.

In some embodiments, the method also includes performing tumor fraction estimation based on one or more weighted signals derived from plasma of the blood sample. The one or more signals can include at least one of: a maximum/average SNV signal, the aneuploidy signal, or a methylation signal. The aneuploidy signal may bes determined by: obtaining copy number data for the plasma of the blood sample; fitting a Gaussian Mixture Model (GMM) to the copy number data, the fitting of the GMM generating a plurality of peaks distributed around a central peak; and inferring a tumor fraction from distances between the plurality of peaks from the central peak.

In some embodiments, a method of performing copy number noise reduction for a batch of a plurality of blood samples includes: obtaining sequencing data for the plurality of blood samples; averaging raw coverages per gene per sample in the sequencing data, the raw coverages measured at a center of each of a plurality of probes; normalizing the normalized coverages to a median gene-level coverage; filtering the normalized coverages for well-behaved probes; creating a model for each sample to predict sample-normalized coverage for guanine-cytosine (GC) content of probes; normalizing the sample-normalized coverages to expected values from the models corresponding to the samples to determine per-sample GC normalized coverage; removing, for all of the plurality of samples, probe values that deviate from the expected value by at least a threshold amount for at least one of the plurality of samples; measuring a median per-sample GC normalized coverage for each probe across the batch; normalizing the per-sample GC normalized coverage to generate expected GC normalized values; and calculating probe copy numbers from the expected GC normalized values.

In some embodiments, a method for genomic profiling of plasma-derived circulating tumor DNA (ctDNA) includes: obtaining a mixture including plasma from a blood sample of a subject, the mixture including cell-free DNA (cfDNA) from the subject; performing processing steps on the mixture to produce a sequencing library, wherein at least some of the processing steps including adding a corresponding set of Quantitative Counting Template (QCT) molecules to the mixture; sequencing the sequencing library to produce sequence reads; performing quality tracking based on a subset of the sequence reads that correspond to the QCT molecules; and responsive to the quality tracking indicating success of the processing steps, constructing a tumor mutational profile for the subject based on the sequence reads. The method may also include estimating a tumor fraction of the ctDNA, with the tumor mutation profile being further based on the tumor fraction.

In some embodiments, a method of genomic profiling of circulating tumor DNA (ctDNA) includes: extracting buffy coat DNA from a blood sample; performing multiplex PCR or ddPCR on the buffy coat DNA for a first set of mutations; identifying, from sequencing data for the first set of mutations, one or more mutations present in the buffy coat DNA; analyzing plasma extracted from the blood sample a hybrid capture method that interrogates millions of base pairs; generating a tumor mutation profile that reports mutations detected by the hybrid capture method that were not present in the buffy coat DNA. The first set of mutations may include 1-10 mutations.

### BRIEF DESCRIPTION OF THE DRAWINGS

FIG. 1A is a high-level overview of an assay workflow for generating sequencing data including sequence reads of circulating tumor DNA, according to one embodiment.

FIG. 1B is a high-level overview of a bioinformatics workflow for generating genomic profiling of circulating tumor DNA, according to one embodiment.

FIGS. 2A and 2B are a set of charts illustrating focal copy number information that distinguishes gene-level changes in copy number from chromosomal arm aneuploidy for amplifications and losses, according to one embodiment.

FIGS. 3A-E are a set of charts illustrating that an embodiment of the assay can detect pathogenic and actionable mutations in a large majority of samples across cancer types.

FIGS. 4A-C are a set of charts illustrating that an embodiment of the assay shows concordance with orthogonal liquid biopsies with increased sensitivity below 0.4% Allele Frequency (AF).

FIG. 5 illustrates the structure of a QCT molecule, according to one embodiment.

FIG. 6 shows concordance of VAF with and without hcQCT addition, according to one embodiment.

FIG. 7 is a histogram illustrating the distribution of instances of measure copy number for plasma-extracted cfDNA, according to one embodiment.

FIG. 8 illustrates the estimated in-tissue versus plasma copy number over time for two genes for a patient, according to one embodiment.

FIG. 9 illustrates the estimated TF and max VAF over time for a patient, according to one embodiment.

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### DETAILED DESCRIPTION

Over the past decade, oncologists have increasingly utilized genomic profiling of tumors via Next Generation Sequencing (NGS) to make better-informed treatment decisions. The intersection of precision oncology diagnostics and targeted therapies have revolutionized cancer treatment by tailoring interventions to the unique biology of each patient's disease. The inherent variability in cancer presentation and behavior underscores the importance of knowing the somatic mutational profile of each patient. Such knowledge empowers oncologists to make informed decisions, increasing the likelihood of selecting the most effective treatment. This approach not only improves health outcomes and quality of life for patients but also enhances the overall efficiency of the healthcare system.

Precision oncology and targeted therapies have demonstrated the effectiveness of tailoring oncology treatments to the biology of each patient's disease. The presentation and behavior of cancer can vary greatly from patient to patient. Knowing the cancer somatic mutational profile of each patient assists oncologists to make well-informed decisions at the treatment selection step. This increases the chances of selecting the most effective treatment for individual patients and improves health outcomes, quality of life, and the efficiency of the health care system. Likewise, better informed decisions can reduce the chances of subjecting patients to ineffective treatments due to the presence of resistance mutations. Ineffective treatments lead to unnecessary side effects, financial costs, and negatively impact the patient's prognosis. For patients with late-stage cancer, it is even more important that their first-line of treatment is effective and specific. A current deterrent to making well-informed treatment selection decisions is the lengthy process of tissue testing and the difficulty or inability to obtain sufficient tumor samples in 30%-40% of the biopsies.

Circulating tumor DNA (ctDNA) levels can be highly variable, depending upon a range of factors, including cancer type, stage, size, and location of the tumor. As such, even large, late-stage cancers may have low variant allelic fractions (VAFs) due to the biology of the tumor. The presence of low allele fractions in plasma does not necessarily mean low amounts of the mutation in the tumor itself but may rather be a reflection of low ctDNA shedding into the bloodstream. Nonetheless, tumors with sensitizing mutations at low VAFs are no less susceptible to targeted therapies when properly identified. Recent studies in NSCLC have shown that as many as 25% of somatic mutations were found below 0.2% VAF, however the limit of detection of the currently available commercial assays are reported to be at 0.2% or greater. However, estimating tumor fraction and inferring copy number alterations (CNAs) from a cell-free DNA (cfDNA) liquid biopsy is challenging due to SNV heterogeneity and potential lack of SNV signal. Therefore, there is an unmet clinical need to be able to identify somatic mutations below a 0.2% VAF to identify more patients who can benefit from targeted therapy.

### Overview

Various embodiments of the disclosed assay capture circulating tumor DNA (ctDNA) in circulating cell-free DNA (cfDNA) extracted from venous blood samples to construct a unique and accurate tumor mutational profile for each patient. The tumor mutational profile contains valuable information on single nucleotide variants (SNVs), insertions and deletions (indels), copy number variations (CNVs), and fusions. With a liquid biopsy and next-generation sequencing approach, results are obtained non-invasively and rapidly. The assay offers the opportunity to make decisions that are better-informed, quicker, and more accurate.

Somatic mutational profiling from liquid biopsies have already been shown to have good correlation with tissue in advanced stage patients, with the added value of being less invasive, less subject to tumor heterogeneity, have faster turn-around times, and can be used when tissue biopsies are difficult or impossible to obtain. The clinical utility of somatic mutational profiling assays has already been shown in multiple cancer types and has already been implemented in NCCN guidelines for NSCLC and mBC. However, the utility of currently available liquid biopsy assays is constrained by poor limits of detection. At first contact with a patient, large panels lose considerable accuracy at low allelic fractions, resulting in no calls for some patients. Alternatively, other panels will require the addition of tissue samples to verify results and create customized ddPCR panels for future longitudinal monitoring, which is an impossibility for patients with no available tissue. Various embodiments of the disclosed assay overcomes these issues by utilizing an 84-gene panel with a limit of detection of 0.13-0.16% allele fraction for SNVs and indels, 0.25%-0.40% for fusions, 2.10-2.16 for Copy Number amplification, 1.7-1.9 copies for Copy Number loss, and 0.07-0.40% allele fraction for MSI.

The assay is a robust, efficient, assay designed to address the unmet need of improved sensitivity and therefore has the ability to improve patient outcomes. The assay builds upon existing clinically validated somatic mutational profiling technology with an improved limit of detection. This improvement provides more precise quantification of somatic mutational profiles, and thus allows for greater discovery of actionable mutations, thereby finding more patients who are likely to benefit from targeted therapies. The assay is CLIA-validated, has shown clinical validity through high concordance with existing assays on the market across multiple cancer types, and has improved clinical utility by providing more actionable data to improve patient management and clinical decision making.

The assay is a plasma-based comprehensive genomic profiling test for solid tumors. In one embodiment, the assay detects actionable mutations, including SNVs, indels, CNVs and fusions across 81 genes. In another embodiment, the assay detects the original 81 genes as well as providing hotspot SNV coverage for two genes, and covering NTRK2 as an additional fusion gene for a total of 84 genes. The assay may also provide coverage to enable reporting of microsatellite instability (MSI) status and improve performance for CNV reporting as well as implementing hybrid-capture QCT (hcQCT) technology for process quality monitoring improvement.

By using Quantitative Counting Template (QCT) technology, various embodiments of an assay can track the yield of multiple processing steps, providing higher sensitivity by increasing the number of decoded molecules after sequencing. In addition, the assay employs chemistry and panel design optimizations, as well as novel algorithms for SNV, indel, CNV, fusion, and MSI calling, to increase sensitivity and specificity.

In view of this, the performance of the assay may surpass that of other commercially available tests for multiple mutation types, detecting more pathogenic and actionable mutations at low tumor DNA fraction while maintaining similarly high specificity. In one embodiment, analytical validation determined limits of detection of 0.15% VAF for SNVs and indels, 2.11 copies for copy number amplifications, 1.8 copies for copy number losses, 0.30% VAF for fusions, and 0.07% tumor fraction for MSI.

The assay was also determined to have clinical utility, with a study of clinical samples (N=481) from a variety of tumor types yielding <2% of reports with 0 mutations detected. A head-to-head comparison with other commercial liquid biopsy assays on a subset of the clinical samples (N=182) showed that the assay detected 50.8% additional pathogenic SNV/indel mutations and 109% additional CNVs, leading to a higher proportion of patients with at least one pathogenic mutation detected (89%) compared to comparators (80%).

These results demonstrate that the assay is an innovative and high-performing diagnostic for treatment selection in oncology, leveraging quantitative approaches in process design, chemistry, and bioinformatics to offer superior analytical and clinical performance compared to the current landscape of liquid biopsy CGP tests. In addition, analysis showed that the proportion of mutations derived from Clonal Hematopoiesis was similar to that of comparator assays, confirming that the additional mutations are almost entirely tumor-derived.

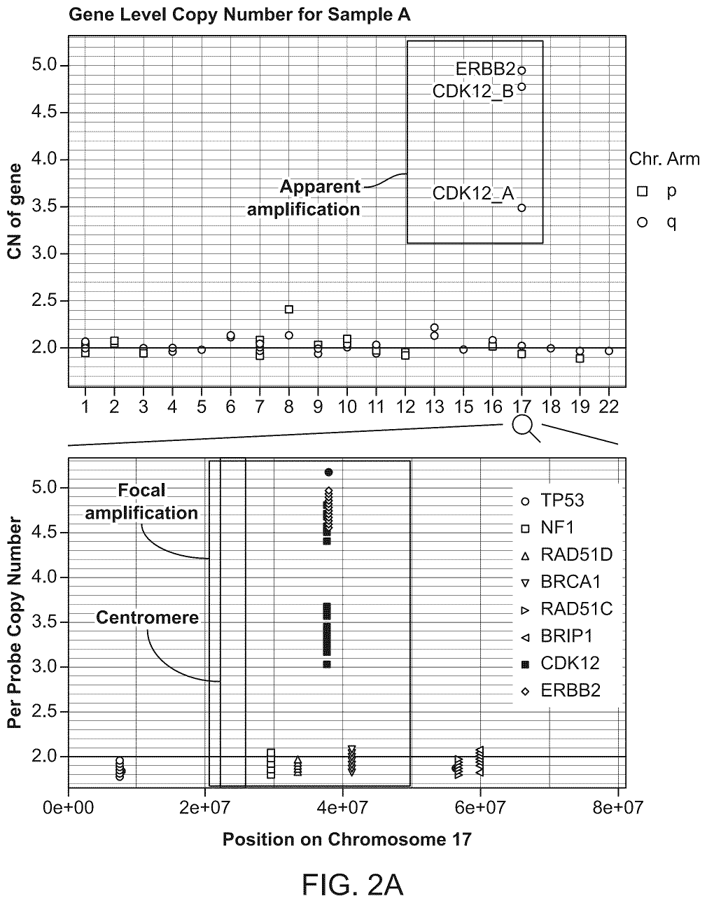

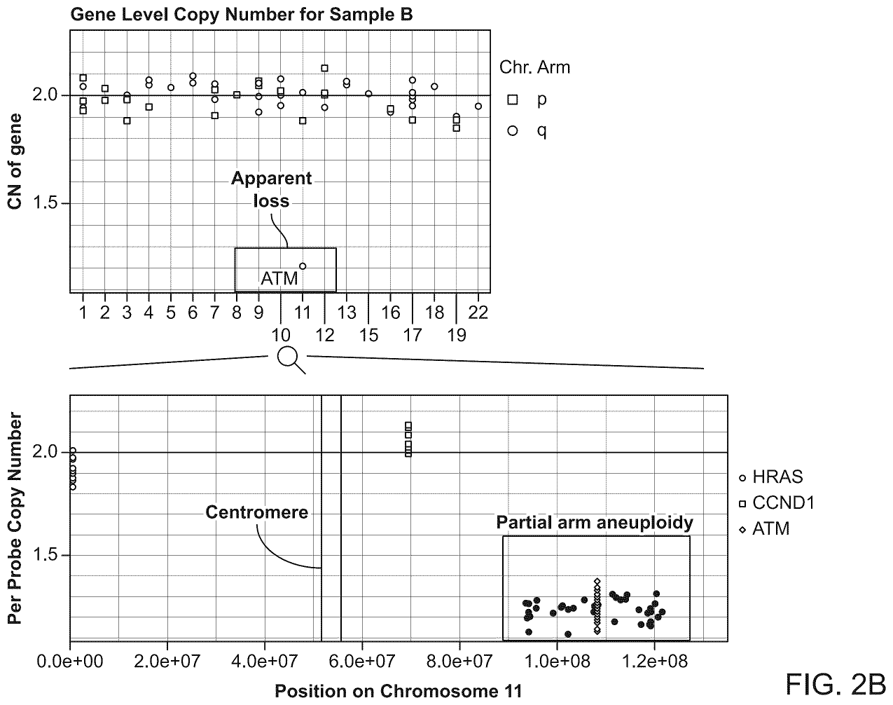

In one embodiment, the intended use population of the assay includes all patients with advanced or metastatic solid tumors. This test can be utilized upon diagnosis to identify targetable mutations and can be used throughout the patient journey to identify tumor evolution or resistance whereby the somatic mutational profile would change. This test can be used in patients who have inaccessible tumors (due to location or patient health), or patients who have insufficient tissue for sequencing analyses. If the test detects actionable mutations in the patient sample, these can be used to for appropriate targeted therapy. The results of the test can also inform if a treatment option is no longer viable due to resistance mutations and inform alternative treatment strategies if new mutations are detected. The test provides information on actionable mutations with a lower limit of detection compared to existing tests. This allows for better detection of patients with targetable mutations, thus providing more precise patient care. FIGS. 2A through 4 illustrate the improved performance that can be provided by embodiments of the assay with reference to various metrics and examples.

FIG. 2A illustrates the focal copy number measured by the assay for various genes, according to one embodiment. The upper, zoomed out portion of FIG. 2A shows chromosome 17 is present in the sample at approximately the expected amount (with a copy number close to 2). However, the lower portion of the chart, which zooms in n the data for Chromosome 17, shows that genes CDK12 and ERBB2 are present at elevated copy numbers. Thus, this illustrates that the assay can distinguish between chromosomal arm aneuploidies and the presence of specific genes at elevated copy numbers. Similarly, FIG. 2B illustrates that the assay can distinguish a copy number loss for a large chromosomal portion versus a focal copy number loss of one (or a small number of) genes.

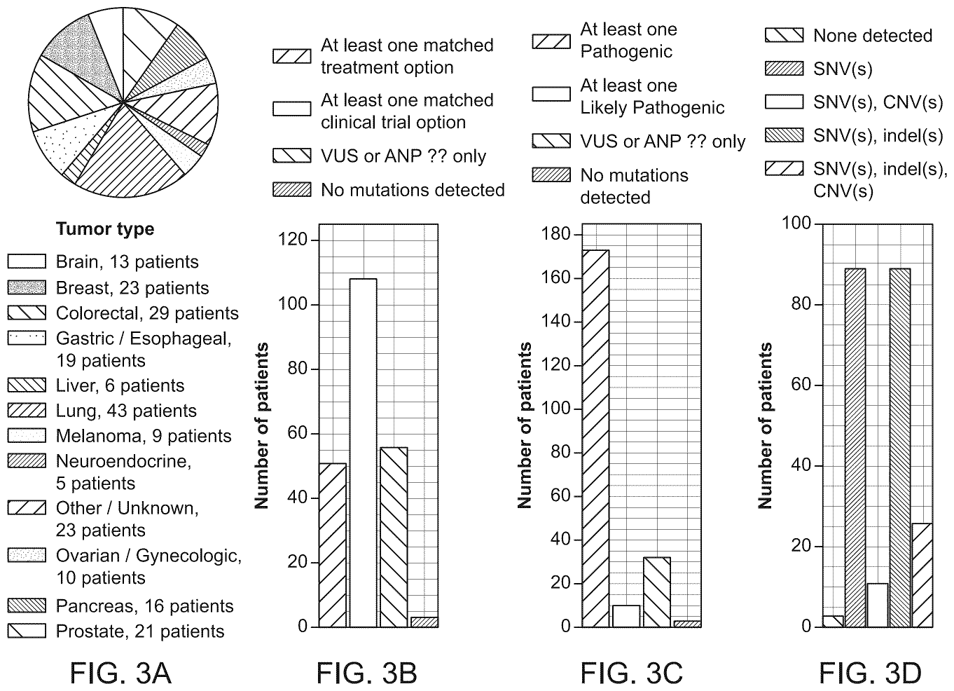

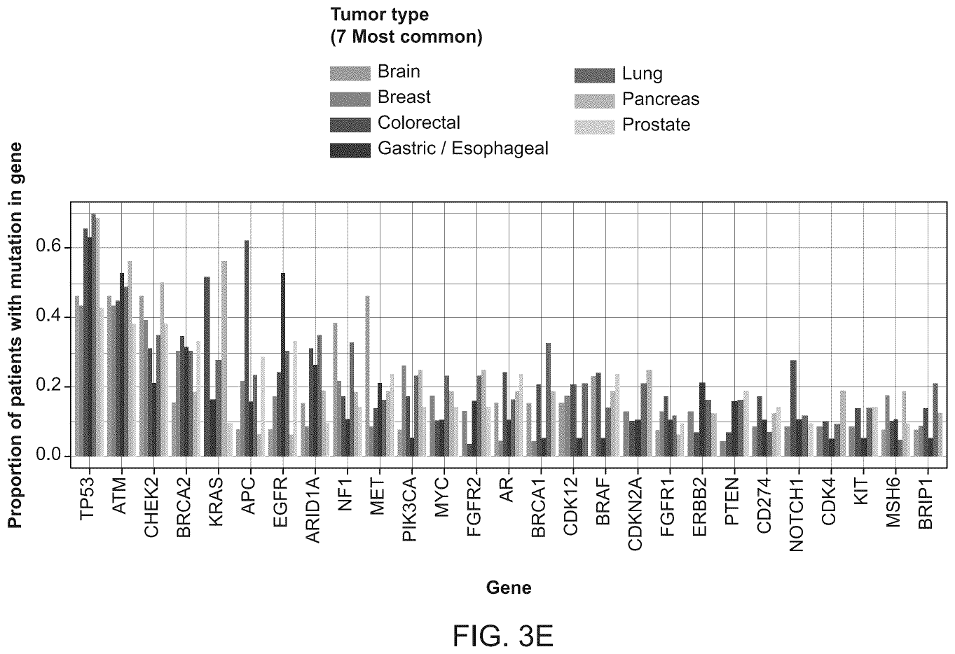

FIGS. 3A-E illustrate results from applying an embodiment of the assay to blood samples taken from a cohort of patients with a variety of cancer types, including brain cancer, breast cancer, colorectal cancer, gastric/esophageal cancer, lung cancer, pancreatic cancer, and prostate cancer. It can be seen from FIGS. 3A-E that the assay successfully detected actionable mutations in a range of genes that are linked to the different types of cancer. Consequently, the assay successfully matched a majority of the cohort to at least one treatment or clinical trial option.

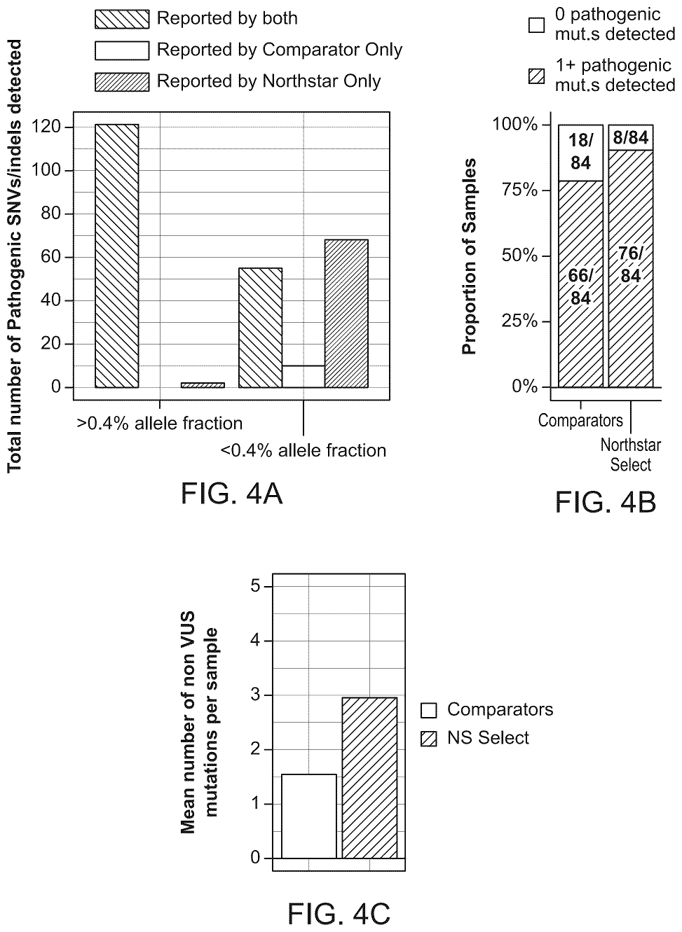

FIGS. 4A-C show a comparison of an embodiment of the instant assay to a comparator assay. As shown in FIG. 4A, the instant assay and comparator assay had largely comparative performance for mutations with >0.4% allele fraction, with the instant assay detecting a small number of mutations missed by the comparator assay. However, for mutations with allele fractions <0.4% the instant assay had significantly better performance, with the instant assay detecting almost seven time more mutations that were missed by the comparator assay than vice versa. As shown in FIG. 4B, the instant assay successfully detected at least one pathogenic mutation in 76 of 84 samples versus the comparator assay only detecting pathogenic mutations in 66 of the 84 samples. Similarly, as shown in FIG. 4C, the instant assay detected almost twice as many Variant of Uncertain Significance (VUS) mutations per sample on average than the comparator assay.

### Assay Design

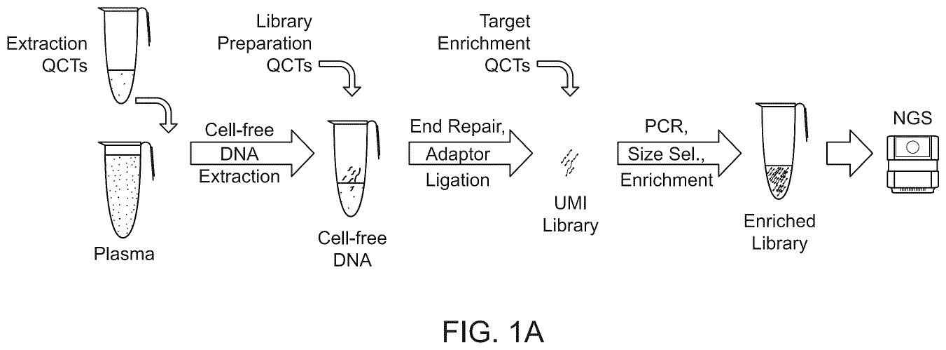

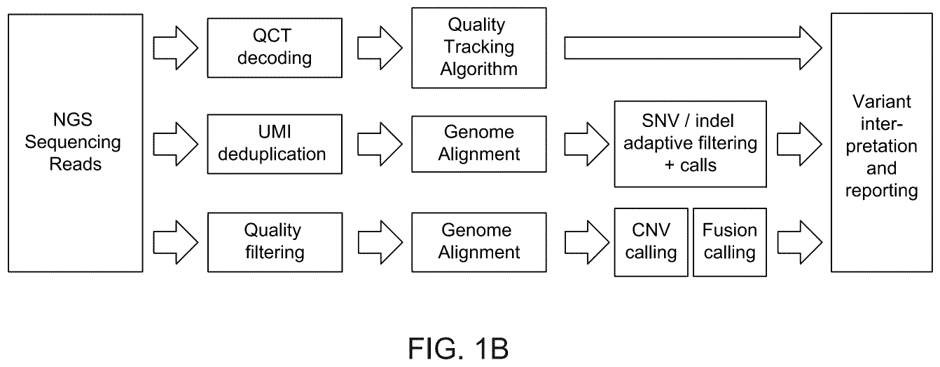

In one embodiment, a comprehensive genomic profiling assay reports multiple classes of oncogenic mutations using a highly optimized custom hybrid capture library preparation approach and QCT technology paired with a curated panel. The assay may use hybrid capture quantitative counting templates, referred to as hcQCTs. The addition of hcQCTs provides robust internal spike-ins that allow for detailed, in-depth analysis of assay performance including assay performance drifts, trends in extraction yield, conversion efficiency, the target enrichment process, operator error, and polymerase error. hcQCT metrics provide a step-by-step orthogonal measurement in tracking assay performance and assist with troubleshooting when necessary. The laboratory, bioinformatics, and reporting workflow is displayed in FIG. 1.

Generally, sets of QCT molecules are added at some or all of a plurality of steps performed on a biological sample (e.g., plasma from a patient) as preparation for performing sequencing. In the embodiment shown in FIG. 1, extraction QCT molecules are added to plasma from a patient and cell free DNA (cfDNA) is extracted. Library preparation QCT molecules are added to the extracted cfDNA and end repair adapter ligation is performed to form a tagged library. Target enrichment QCT molecules are added to the tagged library and a PCR process is performed with size selection and enrichment to form an enriched library. The enriched library is then sequenced using NGS to produce sequence reads. Because different sets of QCT molecules are added at different steps, analysis of the relative amounts of the different types of QCT molecules can be used to identify and evaluate potential failures or other errors and either correct for those issues or determine which steps of the test should be repeated.

Analysis on the sequence reads includes performing QCT decoding, molecule deduplication, and quality filtering. In one embodiment, the analysis including extracting QCT reads from fastqs and calculating unique QCT counts after performing molecule deduplication. Because QCTs are added in several steps of the assay, QCT counts corresponding to the specific QCTs added at different steps can be used for quality control. A low QCT count for a particular QCT molecule can indicate an issue with the corresponding step in the assay. QCT error rates can reveal sequencing errors, dilution issues, and contamination. Other QC metrics unrelated to QCTs include genomic equivalents, yield, sequencing saturation, and on target rate. A quality tracking algorithm along with a genome alignment process with SNV/indel adaptive filtering and calls as well as CNV calling and fusion calling provides variation interpretation and reporting.

Non-QCT reads are processed to generate Binary Alignment Map (BAM) files from which coverage metrics are calculated and variant calling is performed. In one embodiment, SNV/indel analysis includes generating a Variant Call Format (VCF) and adaptive filtering is performed to remove false positives. The adaptive filtering can include using custom scripts with empirically derived error rates, blacklists, and whitelists for specific locations and base flavors on the genome. The CNV analysis includes using peak finding algorithms and rounds of intra and inter sample normalization to perform calling. The CNV analysis may include guanine-cytosine (GC) bias correction. Many genes have focal copy number information obtained via surrounding probes, and the CNV analysis can differentiate between focal and local aneuploidy for those genes (e.g., as shown in FIGS. 2A and 2B). The MSI calling uses a custom bioinformatics algorithm that examines an MSI indel specific VCF filtered by removing inherently noisy sites in negative samples. Fusion calling is performed by analyzing the BAM file for supplementary alignments. To improve specificity, empirically derived scripts can remove likely false positives, correct the orientation of the fusions, or both.

In the clinical lab, sequencing depth can reach a median probe coverage of over 40,000×, with over 100 million paired-end reads per sample. In one embodiment, the custom probe panel covers SNVs and indels in the full coding sequence for 40 genes and hotspots for 44 genes, CN amplifications in 19 genes, CN deletions in 5 genes, fusions in 9 genes, and MSI status for every sample. In the bioinformatics pipeline, a deduplication and error correction approach is combined with adaptive and static thresholding to reduce the number of SNV and indel false positives by over 500×. In addition, copy number data was optimized via a combination of correcting for technical noise and GC skew, and the removal of differential saturation effects, for a net reduction in copy number noise of 4×. Together, these chemistry and bioinformatics approaches can enable detection of actionable mutations from as low as 5 ng of input cfDNA, and at VAFs as low as 0.05%, or 2 mutated molecules out of 4000 total molecules.

### Hybrid Capture Panel Design

In one embodiment, the assay NGS panel can detect SNVs and indels across 150.7 kilobases on 82 genes, of which 40 have whole coding sequence and splice sites coverage while the others have hotspot coverage. The panel is also designed to cover copy number amplification for 19 genes and copy number loss for 5, gene fusions of 9 genes, and MSI status, bringing the total targeted coverage to ˜255 kilobases. The coverage of the assay was designed to prioritize detection of mutations that are either currently actionable via treatment designations or clinical trials, associated with resistance, or known to be pathogenic. Non-actionable pathogenic mutations have potential utility via better understanding the biology of the tumor, or potential future actionability as further therapies progress to trial and eventual approval. Probe placement was optimized to improve diagnostic yield, with increased probe density around actionable point mutations and on fusion genes. In other embodiments, the NGS panel detects SNVs and indels across different ranges of kilobases on different numbers of genes.

### Ligation and Enrichment Efficiency

The performance of four different commercially available library preparation kits was compared on cfDNA samples and gDNA samples designed to mimic the quantity and size distribution of cfDNA. The conversion rate was assessed via qPCR, Qubit, and Tapestation, and in follow-up studies using capillary electrophoresis with fluorescent DNA fragments and NGS. Incubation step length, bead purification concentrations and protocols, and reagent volumes were also optimized. The final optimized protocol had >90% ligation efficiency for 150 bp DNA fragment inserts and 80% for 180 bp fragment inserts, providing a high conversion rate from cfDNA to sequenceable library. The optimized protocol also increased library complexity, the highest unique molecule yield given a certain sequencing depth, which was confirmed with NGS. Finally, a post-ligation double-sided size-selection step was introduced and optimized to enrich for cell-free DNA based library against fragments originating from genomic DNA contamination from lysed white blood cells.

Hybrid capture pull-down efficiency, coverage uniformity, dropout regions, and target enrichment wash stringency were similarly optimized by altering kit and panel vendors, reagents, and conditions and assessing on and off target capture rates via qPCR and NGS. These optimizations yielded a hybrid capture step with a minimum 80% on target rate across all clinical samples, which includes multiple lots of the reagents and custom probe panel. On target rate is the proportion of reads per sample that align in the close vicinity of the probes in the pull-down panel. Whenever a new lot of the panel is ordered, Quality Control (QC) is performed to ensure a desired on target rate (e.g., 82%+) and high coverage uniformity (e.g., fold80 below 1.4) in a standard negative control sample. This may be in addition to further QC performed by the vendor prior to shipping, which may include performing mass spectrometry on each probe to ensure correct length and quantification of each probe, as well as in house custom NGS functional testing to confirm the panel functions as expected.

### QCT Design

The QCTs used are purposely designed components incorporated within experimental protocols. The QCTs function as unique molecule counting spike-ins, serving to validate the proper execution of laboratory procedures and sequencing methodologies for each analyzed sample. These spike-ins can be used to aid in distinguishing variations in cell-free DNA (cfDNA) yields attributed to both biological nuances and technical fluctuations. Additionally, QCTs are helpful for monitoring polymerase and sequencing error rates and sequencing saturation, ensuring the accuracy of the data being analyzed.

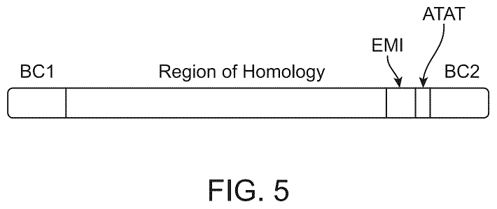

In general, a QCT includes a homologous region that matches (e.g., has sequence identity with) a target genomic region and one or more identifier regions that do not match the target genomic region and enable identification of sequence reads from amplicons generated by amplifying the QCT. In one embodiment, each QCT in the assay includes a 20 nucleotide (nt) designed barcode 1 (BC1), followed by a 95 nt region of homology that is identical to the genomic region and overlaps with a target enrichment probe, a 10 nt fully degenerative synthesized Ns serving as embedded molecular index (EMI), an ‘ATAT’ spacer, and finally a 20 nt designed barcode 2 (BC2). The EMI contains 10 randomized bases of A, C, T, or G to allow counting of the QCT molecules. Each barcode (BC1 and BC2) is designed at least 4 hamming distance away from each other, avoids long (>4) nucleotide repeats, has a GC content between 30-65%, and meets certain Tm thresholds for hairpin and secondary structures. FIG. 5 illustrates the structure of an example QCT molecule. In other embodiments, other QCT structures may be used.

Barcodes may be analyzed in NCBI BLAST to ensure that they do not map to the human genome. QCTs were generated as oligopools with IDT, and double-stranded in house with reverse complement sequences of BC2 and KAPA Hifi polymerase. After double-stranding, the QCTs were quality controlled with tapestation and Qubit, and diluted to working stock solutions.

Different QCTs groups are added at multiple steps of the lab process, and each step's QCTs have step-specific BC1s. Each step, approximately ˜2000 molecules of QCTs from each location were spiked into the samples. Extraction QCTs are added to the plasmas at the start of extraction, library preparation QCTs are added to the cfDNA samples at the start of library prep, and target enrichment QCTs are added at the start of indexing PCR, which is upstream of target enrichment (FIG. 1). The QCT set added at each step includes 10 unique QCT designs matched to 10 genes. The genes are equivalent between the sets but the homologous site within the gene varies.

Due to the stage at which they are added, the target enrichment QCTs may have additional adapters. On the left-hand side, a Truseq Dual Index Read 1 was added along with a self-generated 8 nt unique molecular index (UMI); on the right-hand side, Truseq Dual Index Read 2 was added following a different self-generated 8 nt UMI. These UMIs were generated to have a GC content between 30-70%, not contain long strings repeating a nucleotide, and to be at least 5 hamming distances from the UMI sequences used in the upstream library preparation process.

In one embodiment, each QCT group is obtained as a pool of 10 QCTs (e.g., from a custom oligo vendor) according to a customized design. The QCTs are double-stranded using KAPA HiFi polymerase, and bead cleanup is performed afterwards. These prepared QCTs are diluted and undergo QC before being used in the assay. Experiments during development showed that the addition of QCTs has no negative impact on the accuracy of the assay in calling point mutations.

Dozens of normal samples, and samples with various perturbations were introduced into the assay to determine the normal and abnormal ranges for molecular counting quality control (mcQC) metrics derived from the QCT information. Batch to batch variability was also incorporated into these ranges.

### Assay Methods

Plasma Isolation and cfDNA Extraction

Blood volume for each sample was verified to be >3 mL. The blood was thoroughly mixed via inversion and plasma was isolated via centrifugation. Plasma was stored at −20° C. until proceeding.

Isolated plasma specimens were thawed, vortexed, and briefly centrifuged. Plasma volume was verified to be >1.5 mL. Extraction QCTs were prepared and added to each plasma sample. cfDNA extraction was performed using the Circulating Nucleic Acid Kit (Qiagen) according to provided specifications, which include lysis via Proteinase K, binding to a column membrane, buffer and ethanol washes, and elution via centrifugation.

Extracted cfDNA was quantified using a Qubit 1×dsDNA HS Assay Kit on an Invitrogen Qubit 4 Fluorometer. The minimum sample input was 5 ng and the sample inputs were capped at 50 ng maximum going into each subsequent library preparation reaction.

### Library Preparation

Library preparation was performed using a modified version of the manufacturer-provided protocol for the xGen™ cfDNA & FFPE DNA Library Preparation kit and KAPA HiFi HotStart ReadyMix. Ligation 1 and Ligation 2 incubation times were changed from 15 to 45 minutes. Library preparation QCTs were added at the end repair step to assess library quality, and Target Enrichment (TE) QCTs were added before indexing PCR to assess target enrichment quality. After indexing PCR, a double-sided selection is performed with Kapa HyperPure Beads to remove gDNA contamination.

### Hybridization Capture and Target Enrichment

Six to twelve libraries are pooled together for a single target enrichment reaction at 500 ng input each. The hybridization and target enrichment process was performed with xGen™ Hybridization and Wash Kit (IDT) and KAPA HiFi HotStart ReadyMix according to the manufacturer-provided protocol, using a custom probe panel designed in-house. In one embodiment, the probe panel tests for 120 nucleotide oligos that cover the region being targeted. The panel may use a variable tile overlay strategy with greater tiling (e.g., 2× or 3×tiling) that provides greater sensitivity for actionable loci and gene fusions while using less tiling (e.g., 1× tiling) for loci where less sensitivity is desired. The final enriched pool was quantified using a Qubit 1×dsDNA HS Assay Kit on an Invitrogen Qubit 4 Fluorometer, and average fragment length was determined using an Agilent 4150 TapeStation System.

### Sequencing

Sequencing was performed on an Illumina NextSeq 2000 with P3 flow cells and paired-end 150 sequencing (300 cycles) kits with up to 5% PhiX spike-in with the sequencing libraries, targeting 100 million paired end reads per sample. Base calling was performed with Real-Time Analysis (RTA) 3 software. Raw base call files were demultiplexed and converted to fastqs using DRAGEN BCL Convert (v3.8.4).

### Bioinformatics Methods

### Bioinformatic Process Overview

In various embodiments, the bioinformatics pipeline takes sequencing data (e.g., generated using one of the processes described previously) and processes the sequencing data to perform one or more of: SNV calling, indel calling, SNV false positive filtering, indel false positive filtering, copy number noise reduction, CNV calling, CNV normalization and auto-exclusion, fusion calling, CHIP analysis, and quality control.

In one embodiment, the bioinformatics pipeline begins by performing QCT sequence extraction with SeqKit (v2.1.0) on demultiplexed fastq files for each sample. Custom analysis is then performed on the extracted sequences, as specified below. The remaining non-QCT reads undergo adaptor trimming with Trimmomatic (v0.38).

After adapter trimming, BWA-MEM (v0.7.17) is used for sequence alignment against the reference genome Hs37d5. The resulting aligned BAM files of raw reads are utilized for CNV and Fusion variant analysis, as described further below. The raw read alignment files can be analyzed with Picard (v2.26.11) to collect target enrichment QC metrics.

For SNVs and Indel variants, reads are grouped using any suitable technique to generate individual groups originating from each biological source molecule. Subsequently, the pipeline employs error-correction algorithms to address sequencing errors, PCR amplification artifacts, and other forms of stochastic variation. A single consensus sequence is called from each group. The consensus reads are then remapped using BWA-MEM to update the final consensus read alignment.

The overlapping reads between read1 and read2 are clipped. Subsequently, the resulting deduplicated BAM file undergoes variant calling for SNVs and Indels against a reference genome (e.g., Hs37d5) and downstream false positive filtering as specified in the corresponding section below.

### QCT Analysis

In one embodiment, the QCT quantification is processed from raw fastq files with a custom pipeline using the following steps:

1. The reads containing sequences with <=2 hamming distances from a list of designed BC1 or BC2 in the expected locations are pulled out from the rest of the fastq sequences, and the identity of which QCT group the read came from is identified through BC1 sequence matches.

2. The paired sequences of read1 and read2 are merged and analyzed to identify full QCT sequences and their components.

3. Sequences are filtered based on specified criteria (e.g., merging metric of the paired reads) and low supporting read EMIs are corrected to the abundant EMI with the closest hamming distance to avoid excessive counting due to sequencing or polymerase errors.

4. Unique molecule counts (based on unique species of EMI) and mean read counts per QCT are calculated.

5. Saturation correction is applied to account for potential sequencing saturation.

6. For each sample, mcQC z-scores are calculated for each QCT unique molecule count (after saturation correction) on each location and each QCT group. The mcQC z-score is calculated based on empirically measured normal quality samples and libraries.

7. Base error rates (polymerase and sequencing errors) are computed for each QCT location based on anticipated sequences in QCT components.

The number of unique molecules detected in a sample is greatly affected by sequencing depth, so the saturation correction is performed for every QCT count via the following 2 steps:

$$Saturation Level = 1 - ( \# deduped read / \# total reads )$$

$$Saturation Corrected Molecule Count = \# unique molecules / Saturation Level$$

### SNV and Indels False Positive Filtering and Calling

Variant calling is performed using Vardict against a reference genome (e.g., Hs37d5), specifying custom exonic and splice coverage regions, with a minimum variant read threshold of 2-3 unique variant molecules (depending on the locations). Downstream false positive filtering can be performed to obtain robust variant results for SNVs and Indels, especially for low VAF range mutations, which are prevalent in cancer liquid biopsy samples. Adaptive filtering approaches may be used to account for varying levels of false positives on both a per-base and per-mutation basis, as well as considering unique molecule yield.

In a verification study, over 100 paired healthy clinical samples were run, with each pair of two blood tubes of samples corresponding to the same healthy individual from the same day. The samples were prepared end-to-end, sequenced and analyzed as described above. Any discrepancy in SNV and Indel findings between the paired samples can be attributed to potential false positive detections. Several computational approaches based on observations in these true negatives with reproducibility data constitute the adaptive filtering algorithm.

First, the empirical error rate of polymerase for each substitution (e.g. C>T/G>A substitution errors are known to be more frequent than other substitutions) can be determined by compiling all the discrepant low VD (variant read) mutations in all the paired samples and the substitution error rate (i.e. per base per read substitution false positive probability) can be calculated. The expected error rate can be calculated as p=(observations of mutation type)/(total # of bases involved). For example, for the C>T/G>A mutation type p=(observations of C>T and G>A)/(# of Cs+ # of Gs), in all the samples measured. Second, specific bases with more than two pairs of samples showing the error can be compiled into a ‘graylist’ of mutations with a higher error rate than can be explained by polymerase error alone. For the gray listed mutations, expected error rate can be modeled as p=(observed variant molecules)/(total molecules) in the paired samples analyzed. In the bioinformatics pipeline, a P-value is calculated using a binomial distribution comparing the observed number of mutant molecules, the total molecules detected at the position, as well as the mutation specific false positive probability (error rate, either from panel-wide substitution or the mutation specific gray list). Different p-value thresholds were developed for panel-wide SNV substitutions, gray listed SNVs, and gray listed indels and complex mutations. Observed signals with a P-value above the threshold were filtered out. Finally, panel-wide C>T/G>A SNVs (as empirically determined to be the most prevalent false positive mutations during optimization) with a VAF <0.08% were filtered even if they are below the p-value threshold, with a whitelist of specific actionable mutations are exempted.

In addition to the adaptive filtering approaches, static filters may be applied via include lists or ignore lists for certain mutations. Include-listed mutations will be called regardless of the adaptive calculations if 2 or greater unique mutant molecules are observed, while ignore-listed mutations will never be called. The ignore list may be used to exclude assay and sequencing artifacts that show up so commonly or at such high VAF that they cannot be confidently called in any case.

Typically, these adaptive filters result in ˜20 to 50+ false positive point mutations per sample being removed from calling.

### Copy Number Noise Reduction

In some embodiments, the assay uses a combination of coverage data normalizations and corrections to reduce the technical noise of measuring copy number. The normalizations and corrections account for per-sample and per-batch biases in cell free DNA extraction, library preparation, sequencing saturation, and probe copy number predictiveness. In one embodiment, the normalizations and corrections include: (1) Averaging raw coverage measured at the center of each probe per gene for each sample, and normalizing coverage to the median gene-level coverage. (2) Filtering data for well-behaved probes (meaning probes that produce a response curve for varying concentrations that align with expectations according to one or more metrics), and creating a generalized additive model for each sample to predict sample-normalized coverage per GC content of the probe. (3) Normalizing the sample-normalized coverage to the expected value from the generalized additive model for the sample to determine per-sample GC normalized coverage. Probe values that deviate from the predicted value by a certain z-score threshold are removed from all samples in the batch. (4) Measuring the median per-sample GC normalized coverage for each probe across the batch, and normalizing the per-sample GC normalized coverage to that expected value and multiplying it by two to calculate the probe copy number.

### CNV Calling

In one embodiment, copy number per probe is grouped per gene, and a moving average approach is used to determine which probes are statistically different from the surrounding region by using a z score threshold. These probes with large deviations in at least one sample are excluded on a per batch basis. Thereafter, the moving average approach is used to detect where in each gene there is most likely to be a breakpoint based on the relative noise in the moving average. If the noise at that location is above a threshold, a within-gene breakpoint is called and the gene assignment is split for the probes before and after the breakpoint. Then, copy number is calculated for each gene and split gene fragment by taking the weighted average of the probe copy number. The weights are 1 over the probe CV squared, with the CV for each probe taken across the copy numbers of the probe in all non-highly aneuploidy affected samples, which are determined by bioinformatics (BI) operator review or the autoexclusion algorithm described below.

A center calibration algorithm may be applied to set the two copy genes to an average copy number of two, because the average probe copy of the sample may not be two when the tumor is highly aneuploid or the sample is very noisy. This algorithm disregards X chromosome and low probe count genes, removes the two highest and lowest outliers for each sample, and then excludes genes with copy number above 3 or below 1.5. A third exclusion step removes genes with copy number outside of +0.9 z scores of the mean, with the z score calculated based on the standard deviation taken after the initial outlier exclusions. The filtered data is fed into a weighted maximum likelihood estimator that simulates normal distributions with a CV 1.5× lower than the measured CV of the filtered data, and chooses the mean of the normal distribution with the highest summed probability density of all the observed gene copy numbers as the new 2 copy value. The weight used for each gene copy number is the cubed root of the number of probes in the gene. Gene copy number values are corrected by dividing by the new 2 copy value and multiplying by 2. Threshold and cutoff values for these steps were empirically determined to be the best value for fitting the data based on reviewing over 100 samples.

In some embodiments, the gene copy number distribution is used to perform aneuploidy calibration for each sample, and gene copy number amplifications and deletions are called using adaptive thresholds that account for the aneuploidy patterns. First, highest and lowest non-CNV (HNC and LNC) copy number measurements are determined per sample, via the 5th and 90th percent quantile of the CNs of all low noise genes in the assay. In an aneuploid sample, the HNC and LNC value will typically reflect the level of aneuploidy, landing around tumor copy equivalents of 3-4 copies or 1 copy, respectively, which was observed empirically in pure tumor data. Each sample has multiple adaptive thresholds-signal must be beyond the HCN or LCN by a certain absolute (+0.05 or −0.04 CN) and relative (+2.5/+1.5 for genes with focal information or −0.9 z score) amount, as well as in the reportable range for the assay (<1.9 or >2.1 CN) to be called.

Next, for a subset of CNV reportable genes, including all 5 copy number deletion reportable genes, copy number measurements on probes up- and down-stream of the gene are used to confirm that the copy number deviation is focal, as opposed to resulting from aneuploidy of the chromosomal arm. This focal confirmation dramatically increases the confidence that the mutation is oncogenic.

The sample calibrated CNV calls are manually reviewed by the BI operator for acceptance/rejection in the case of CN signal very close in level to the signal for another gene, a within gene breakpoint detected, an AR (chromosome X) amplification call, or focal information with a low ratio of the focal signal to the signal relative to 2 copies.

For copy number signals that are significantly different from the 2 copy distribution, as determined by having a deviation of the greater of half the HNC deviation or half the LNC deviation, the genes are called as non-CNV CN deviation, and included as a VUS in the report. These calls typically reflect aneuploidy patterns as opposed to focal amplifications or losses.

### CNV Algorithm Normalization and Auto-Exclusion

In some embodiments, the copy number bioinformatics pipeline identifies and quantifies CNVs using a comparative genomic hybridization-like method. Three forms of normalization are applied to control for technical noise and batch effects. First, batch normalization utilizes process-matched positive and negative controls to normalize coverage across probes in each batch. Second, focal sample normalization normalizes coverage against regional genomic variation such as GC bias. Third, genome-wide single sample normalization minimizes coverage bias across all the probes in each individual sample. Thus, the assay controls for bias in each batch, per each sample, and per each region. Changes in copy number are identified with a sliding window-based algorithm, with statistically significant shifts in probe coverage statistics indicating a copy number transition event. Next, intra-gene copy number count transitions are identified, and per-gene copy number for each sample is determined. Per-sample normalization applies a peak-fitting algorithm and determines genome-wide aneuploidy calibration levels. For a subset of CNV reportable genes, including all deletion reportable genes, a coverage-based focal normalization, against the surrounding regions of the chromosome that the genes reside on, is used to separate CNVs that are focal as opposed to resulting from chromosome arm aneuploidy.

After the initial results, the analysis is repeated twice with selected samples excluded in the batch normalization baseline calculation. These excluded samples have aneuploidy and CNVs in the previous analysis result and excluding them generates a more accurate normalization baseline calculation. When many aneuploid or CNV samples are in a processing batch, samples are scored according to their CNV features using weights from a machine-learning model trained on clinical data. Samples with the highest scores are excluded in the repeat analysis.

### Fusion Calling

In some embodiments, SViCT (v1.0.1) is used to call potential fusion breakpoints for structural variants (e.g., gene fusions) based on the raw mapped BAM file with some custom script modifications. SViCT extracts discordant mapped read pairs (which may represent fusion breakpoints), including one-end anchors and soft-clipped split reads, and groups them into clusters based on soft-clip or one-end location. The read ends are assembled into contigs (intervals), subsequently indexed, and reference k-mers are mapped to each contig. The intervals are then joined using a combination of graph and greedy algorithms to identify specific structural variant signatures.

SViCT is an algorithm with a strong emphasis on sensitivity in the context of liquid biopsy; however, the results are often not very specific, as it returns a potential list of thousands of breakpoints in any given sample. Downstream filtering and fusion orientation correction scripts are implemented to computationally remove non-specific breakpoint calls. First, the VCF output of SViCT is filtered to only include fusion variants (bnd) with a support score greater than 15, and at least one side of the breakpoint is inside the genes covered for fusion by the assay. Second, a deduplicated BAM file with any supplementary alignment information (referred to as the supplementary BAM file) is generated by filtering the deduplicated BAM file to include only reads with a Supplementary Alignment tag. Afterwards, the filtered VCF is corrected by programmatically examining the supplementary alignment BAM file at the potential breakpoint detected by SViCT and removing false positive SViCT calls without 2 unique supporting unique reads of the breakpoint in the supplementary BAM. The orientation of the fusion calls represented in VCF 4.2 format is also corrected by examining the coverage patterns of the supplementary BAM file on both sides of the breakpoints.

The final fusion breakpoints are interpreted together with other variants as described below.

### Filtering Mutation Detection Using Buffy Coat Analysis

Detecting the presence of a mutation from a plasma sample does not necessarily reveal the source of that mutation. In evaluating treatments options, it is significant whether a detected mutation is tumor-derived or not. This may be addressed by analyzing the buffy coat in the blood sample from which the plasma was extracted. If the same mutation is present in the buffy coat, this indicates that the mutation is not tumor-derived. Mutations that are detected in both the plasma and the buffy coat may be filtered out and not reported. In some embodiments, a hybrid capture approach interrogates many (e.g., millions of) base pairs from the plasma of a blood sample and the results of the hybrid capture approach are used to design a bespoke buffy coat analysis (e.g., using ddPCR or multiplex PCR, etc.) to determine if one or more mutations that were detected in the plasma are also present in the buffy coat from the same blood sample. Mutations can be detected in the buffy coat (and filtered) at low concentrations (e.g., 0.05%-20% VAF).

In some embodiments, the buffy coat is analyzed to detect Clonal Hematopoiesis of Indeterminate Potential (CHIP) mutations, for which several methodologies can be employed. In one embodiment, a digital droplet PCR (ddPCR) approach involves obtaining a patient blood sample, separating the buffy coat, and extracting and fragmenting genomic DNA. This DNA is then mixed with a PCR master mix, specific primers, and Taqman probes, partitioned into ˜1 nL droplets, and subjected to endpoint thermal cycling. A droplet reader measures fluorescence to classify each droplet as positive or negative, calculating the variant allele frequency (VAF) to confirm CHIP mutations by comparing results with liquid biopsy assays.

Alternatively, a Comprehensive Genomic Profiling (CGP) approach evaluates numerous gene alterations in a single assay, offering a broader overview that helps identify CHIP mutations by comparing genetic profiles from tumor tissue and blood samples. Additionally, reflexing to small multiplex PCR reactions based on potential CHIP variants can offer efficient screening for multiple CHIP-related mutations at once with customizing to each sample's mutation profile.

In another embodiment, a multiplex PCR approach is used. Primers for different CHIP mutations of interest are added to a sample from a subject and the resulting mixture is co-amplified in a multiplex PCR operations. NGS is then used to determine the presence or absence of each mutation of interest. In some embodiments, QCT molecules that have a target matching region (with sequence similarity to a corresponding mutation of interest) and an identification region (that has sequence dissimilarity to the corresponding mutation and instead includes an identifier of the QCT molecule) may be used to enable quantitative measurement of the amount of each mutation present.

The CHIP reflex workflow with enzymatic shearing (or mechanical shearing) can be launched and run the CHIP assay on the buffy coat gDNA. This assay can run on patients' buffy coat to assess whether somatic variants present in plasma are tumor-derived.

### Quality Control

In one embodiment, QCT metrics as described above (z-scores, sequencing saturation level, and base-wise error rate) for each QCT group are documented in mcQC metrics for each sample for process tracking and process quality control use, typically mcQC z-scores below-3 are flagged as potential process failures.

Further QC metrics are generated and monitored as part of the bioinformatics pipeline. Sequencing error rate can be calculated in the pipeline. Mean GE is the mean genomic equivalent per sample, excluding QC fails. The mean OTR is the mean on target rate, or the percentage of aligned reads that were located on or near the baited (probe) regions. The passing criteria per batch is defined as having a sequencing error rate <1%, mean on-target-rate (OTR)>80%, and mean reads/sample >100M. The per sample QC pass criteria is determined by sample mean GE >600, sample OTR >60%, and sample sequencing depth >60M reads.

In addition, positive and negative controls are run in each clinical run. The Positive control is a 3rd party synthetic reference material also containing spiked-in sheared tumor gDNA with known CNAs. The negative control is a pool of cfDNA from healthy donors. New pools are generated and validated upon depletion of the negative control. The new negative control is run in a batch with the old negative control and analyzed for quality control metrics including coverage uniformity, on target rate, and negative mutation calls.

### Analytical Validation and Clinical Validation

### Analytical Sensitivity and Specificity

To assess the analytical performance of the assay, contrived samples for SNVs, indels, and CNVs were created by mixing one part genomic DNA (gDNA) extracted from 10 separate cancer tissue samples with nine parts of the corresponding buffy coat gDNA by mass. The resulting contrived samples had SNVs, indels, and complex mutations at low percentage variant allele fractions (VAF) of 0.8-10%, as well as CNVs. 5 of the samples were run with 2 replicates, for a total of 15 specimens. In order to assess MSI sensitivity, contrived MSI-H samples were similarly prepared to target approximately 1% tumor fraction, accounting for the tumor purity. MSI-H status was confirmed via PCR or IHC by the biobank. The reference samples with known genetic alterations, as well as 2 negative control contrived samples, were also included. The reference materials contain many low VAF SNV and indels likely generated from the synthesis process, and are therefore excluded from calculation of specificity on small variants or MSI.

Sensitivity for each mutation type was calculated as the fraction of correctly identified mutations out of the number of ground truth positives. 58 samples from 39 individual healthy donors were run with Northstar Select to assess the false positive rate in clinical samples on a panel-wide base-wise basis. Confidence intervals were calculated using Clopper-Pearson exact estimation.

### Limit of Detection

An LOD study was performed for small variants such as SNVs and indels using two reference materials. Horizon Discovery Multiplex cfDNA reference standards (HD780) containing 8 variants (6 SNV and 2 Indel) confirmed by digital droplet PCR (ddPCR) were run at 0.15% and 0.25% VAF (1 and ˜1.5×LOD) at three different inputs: 10 ng, 30 ng, and 50 ng. Twist cfDNA pan-cancer reference v2 contains 111 variants (71 SNV and 40 Indels) over 42 genes, confirmed by NS550 sequencing and ddPCR. These were diluted in cfDNA to 0.15% and 0.25% VAF, and run at 10, 30, and 50 ng inputs (Table 3a).

TABLE 3a
SNV/Indel LOD at 3 inputs and 1x and 1.5x LOD

| | DNA | VAF | | | DNA | | |
|---|---|---|---|---|---|---|---|
| | Quantity | 0.15% | | | Quantity | VAF | |
| Variant | (ng) | VAF | 0.25% | Variant | (ng) | 0.15% | 0.25% |
| SNVs | 10 | 297/415 | 344/415 | SNVs | 10 | 71.57% | 82.89% |
| | 30 | 386/415 | 412/415 | | 30 | 93.01% | 99.28% |
| | 50 | 400/415 | 414/415 | | 50 | 96.39% | 99.76% |
| Indels | 10 | 159/220 | 186/220 | Indels | 10 | 72.27% | 84.55% |
| | 30 | 207/220 | 219/220 | | 30 | 94.09% | 99.55% |
| | 50 | 209/220 | 220/220 | | 50 | 95.00% | 100.00% |

An LOD study was performed for CN amplification and CN losses using well-characterized reference materials. Two cell-line reference materials from ATCC, SNU-16 and NCI-H2170, both containing CDKN2A losses and two further reference materials, Horizon HD836 Prostate Cancer Panel cfDNA Reference Standard and HD837, both containing PTEN losses confirmed by Horizon Discovery via ddPCR, were chosen for the CN loss study. For copy number amplifications, two ddPCR confirmed standards from Seracare, Seraseq CNV Breast Mix (containing EGFR) and Seraseq Lung and Brain CNV Mix (containing ERBB2 and MET), were assayed. Horizon Discovery HD836 also contains a ddPCR confirmed AR amplification, and thus was run as part of the CN Amplification LOD study. This totaled 6 unique reference materials tested. The reference materials were diluted in NA12878, to target LOD of 2.11 CN and 2.15 CN for amplification, and 1.8 and 1.5 CN loss. Each standard was tested at three different input masses of 10, 30, and 50 ng, with five replicates at each condition. This totaled eight expected CN variants in six genes (2×CDKN2A, 2×PTEN, 1×MET, 1× AR, 1×EGFR, 1×ERBB2) (Table 3b).

TABLE 3b
Copy Number LOD Study for Amplification and Loss

| | DNA | | | | | DNA | | | |
|---|---|---|---|---|---|---|---|---|---|
| | Quantity | | Copies | | | Quantity | Gene | Copies | |
| Variant | (ng) | Gene | 2.11 | 2.15 | Variant | (ng) | (Sample) | 1.8 | 1.5 |
| Copy | 10 | EGFR | 5/5 | 5/5 | Copy | 10 | CDKN2A_1 | 5/5 | 5/5 |
| Number | | MET | 5/5 | 5/5 | Number | | CDKN2A_2 | 5/5 | 5/5 |
| Amp. | | ERBB2 | 5/5 | 5/5 | Loss | | PTEN_1 | 5/5 | 5/5 |
| | | AR | 5/5 | 5/5 | | | PTEN_2 | 5/5 | 5/5 |
| | 30 | EGFR | 5/5 | 4/4 | | 30 | CDKN2A_1 | 5/5 | 5/5 |
| | | MET | 5/5 | 5/5 | | | CDKN2A_2 | 5/5 | 5/5 |
| | | ERBB2 | 5/5 | 5/5 | | | PTEN_1 | 6/6 | 5/5 |
| | | AR | 5/5 | 5/5 | | | PTEN_2 | 5/5 | 5/5 |
| | 50 | EGFR | 5/5 | 5/5 | | 50 | CDKN2A_1 | 5/5 | 5/5 |
| | | MET | 5/5 | 5/5 | | | CDKN2A_2 | 5/5 | 5/5 |
| | | ERBB2 | 5/5 | 5/5 | | | PTEN_1 | 5/5 | 5/5 |
| | | AR | 5/5 | 5/5 | | | PTEN_2 | 5/5 | 5/5 |

To assess the LOD of fusions, six synthetic fusions were designed. Oligo pools were generated with a sliding window across each of the breakpoints. The length of the window mimicked the length of cfDNA. An SNV was designed near the fusion breakpoints to accurately gauge the allele fraction. Each oligo pool was spiked into a buffy coat at various allele frequencies, targeting 0.1-2% and 20 replicates were through the assay (Table 3c). The SNV-INDEL LOD study reference material (Twist cfDNA Pan-cancer) also contains 10 fusion variants in 2 genes. Fusions tested were: TRP-ALK, EML4-ALK×3 breakpoint variants, CCDC6-RET×3 breakpoint variants, KIF5B-RET×2 breakpoint variants, and NCOA4-RET. These were analyzed at the three inputs and two VAFs already described in the SNV LOD study (Table 3d).

TABLE 3c
Fusion LOD Analysis

| Tumor Fraction | 0.15% | 0.30% | 0.66% | 1.54% |
|---|---|---|---|---|
| Positive Expected | 60 | 20 | 20 | 20 |
| Positive Detected NS2000 # | 53 | 20 | 20 | 20 |

TABLE 3d
Fusions present in SNV/INDEL LOD study

| | DNA Quantity | | |
|---|---|---|---|
| Variant | (ng) | 0.15% VAF | 0.25% VAF |
| Fusions | 10 | 1/50 | 3/50 |
| | | (2.00%) | (6.00%) |
| | 30 | 17/50 | 36/50 |
| | | (34.00%) | (72.0%) |
| | 50 | 25/50 | 47/50 |
| | | (50.00%) | (94.00%) |

Eight MSI-H solid tumor tissue samples were ordered from Accio Biobank and confirmed MSI-H via IHC or PCR, along with their paired buffy coats. The LOD was computationally estimated by bioinformatically combining reads from the buffy coat and the tumor in allele fractions of 0.03, 0.05, 0.07, 0.1, 0.15, 0.2, 0.25, 0.50, and 1.00% (Table 3e). To confirm the LOD of MSI, an MSI-H sample was run with the assay at a 30 ng input, along with its paired buffy coat (Table 3f). Both tumor and buffy coat gDNA sample inputs were sonically sheared to mimic cfDNA size distribution. Tumor purity was estimated using the maximum VAF of pathogenic mutations.

TABLE 3e
MSI Analytical Sample LOD Estimation

| Tumor Fraction | 0.03% | 0.05% | 0.07% | 0.10% | 0.15% | 0.20% | 0.25% | 0.50% | 1.00% |
|---|---|---|---|---|---|---|---|---|---|
| Detected | 0 | 14 | 17 | 20 | 20 | 20 | 20 | 20 | 20 |
| Expected | 20 | 20 | 20 | 20 | 20 | 20 | 20 | 20 | 20 |
| Sensitivity | 0% | 70% | 85% | 100% | 100% | 100% | 100% | 100% | 100% |

TABLE 3f
MSI Analytical Sample LOD Confirmation

| Tumor Fraction | 0.07% |
|---|---|
| Positive Expected # | 20 |
| Positive Detected NS2000 # | 19 (95.00%) |
| Positive Detected NovaseqX + # | 19 (95.00%) |

### Limit of Blank (LOB)

58 healthy patient plasmas were run through the Northstar Select assay and assessed for any potential false positives. In 8,750,344 bases, one pathogenic indel and one SNV were detected (Appendix C.). The detection rate of pathogenic alteration was 0% for CNV, MSI-H, and rearrangements.

### Reproducibility, Repeatability, and Robustness

Twenty samples with 42 SNV/Indel variants were run three times within a batch to test repeatability. The same batch of samples were run two more times by different operators on different days to test reproducibility, for a total of five replicates. 38/42 variants were detected in all three replicates within the run, for a repeatability of 90.5%, 40/42 variants were detected in at least two of the three runs for repeatability, and both of the additional reproducibility runs, for a reproducibility of 40/42 (95.2%). Six CNVs were expected, and all 6 were detected and called correctly across the 5 replicates. The assay's robustness was assessed by adding a known interfering substance, hematin, at concentrations ranging from 0.001-10 μM to Twist reference standard DNA. At all concentrations of hematin, GE yield and calling accuracy was unchanged from normal.

hcQCT Inclusion

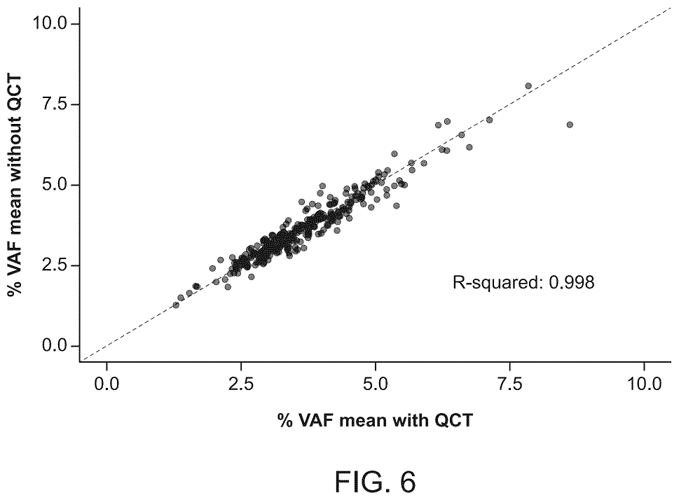

To assess if QCT addition had any effect, samples were tested with and without QCT addition. Each point represents a single SNV or indel variant in the reference material. The plot shows that with (n=4) and without (n=4) QCTs, the positive detection of variants and mean VAF detected do not differ significantly. FIG. 6 shows concordance of VAF with and without hcQCT addition.

### Clinical Performance and Validation

Utilization of mcQC Scores Improves Clinical Workflow

The complex nature of a hybrid capture based liquid biopsy's multi-day workflow leaves room for error that is not easily traceable. Errors may occur at any step of the assay, including extraction, library preparation, and target enrichment. Depending on the nature of the error, this process can be time-consuming. Moreover, if there was a technical error, it is helpful to determine at which step the error occurred. This is because the step that failed determines what step(s) need to be redone. For example, a batch that fails target enrichment can have its libraries re-processed through target enrichment instead of re-processing the samples from the very beginning of the workflow.

In the embodiment shown in FIG. 1A, where hcQCTs are added at three points in the assay, the resulting three groups of sequencing reads (corresponding to sequence reads of amplicons generated from the added hcQCTs) together identify the source of error in failed samples based on the idea that errors negatively affect the yield of upstream hcQCTs, but not the yield of those added downstream. For example, an error in library preparation should be reflected in low yield for both EXT and LP hcQCTs, but normal yield of TE hcQCTs. An error in target enrichment should be reflected in low yield for all three hcQCT groups. On the other hand, if there was low GE recovery of a sample and the yield was normal for all three hcQCT groups, it is most likely that the sample input was low in GE. hcQCTs make it possible for the assay to identify the step at which an error occurred in failed samples. Additionally, the hcQCTs provide methods to independently estimate sequencing saturation and to track sequencing and polymerase error.

Each hcQCT group offers unique and novel information on assay performance. EXT hcQCTs can identify the presence of extraction related errors. Next, LP hcQCTs can identify the presence of library preparation related errors. In samples with low GE recovery, LP hcQCTs can also distinguish technical errors in sample processing from the biological nature of some blood samples with low cfDNA content. TE hcQCTs can identify the presence of hybridization or target enrichment related errors.

The three groups together help to identify the source of error in failed samples based off of the idea that errors negatively affect the yield of upstream hcQCTs, but not the yield of those added downstream. For example, an error in library preparation should be reflected in low yield for both EXT and LP hcQCTs, but normal yield of TE hcQCTs. An error in target enrichment should be reflected in low yield for all three hcQCT groups. On the other hand, if there was low GE recovery of a sample and the yield was normal for all three hcQCT groups, it is most likely that the sample input was low in GE. hcQCTs make it possible for the assay to identify the step at which an error occurred in failed samples.

Additionally, the hcQCTs provide methods to independently estimate sequencing saturation and to track sequencing and polymerase error.

TABLE 4
mcQC scoring enables detection of and response to various failure modes

| | cfDNA | Library | Target | | | |
|---|---|---|---|---|---|---|
| Lab | Extraction | preparation | enrichment | | | |
| QC | mcQC | mcQC | mcQC | Sequencing | | |
| result | result | result | result | QC result | Significance | Followup |
| Pass | Pass | Pass | Pass | Pass | | Accept |
| | | | | | | results |
| Pass | Fail | Pass | Pass | Pass | cfDNA | Repeat |
| | | | | | extraction | plasma |
| | | | | | failure, | isolation |
| | | | | | potentially due | onwards. |
| | | | | | to poor sample | Potentially |
| | | | | | quality | redraw |
| Pass | Pass | Fail | Pass/Fail | Pass/Fail | Library | Repeat |
| | | | | | preparation | plasma |
| | | | | | failure, likely | isolation |
| | | | | | due to | onwards |
| | | | | | divergent | |
| | | | | | conditions for | |
| | | | | | the sample | |
| Pass | Pass | Pass | Fail | Pass/Fail | Target | Repeat |
| | | | | | enrichment | target |
| | | | | | failure, usually | enrichment |
| | | | | | reflecting | onwards |
| | | | | | some deviation | |
| | | | | | in process | |
| | | | | | quality for the | |
| | | | | | entire batch | |

### Clinical Sample Characteristics

Patient blood samples of approximately 10 mL were collected using a 21-gauge straight needle in Streck tubes and mixed with the proprietary preservative reagents by gently inverting. To ensure accurate sample identification, the patient's full name and date of birth were clearly labeled on the tubes. Specimens were securely packaged for transportation within a biohazard bag, cushioned using a gel pack and enclosed in a foil bag within a BillionToOne kit. These prepared kits were sent for transportation using courier services, maintaining strict temperature control during transit. Upon receipt at the laboratory, cleaning of the workspaces was conducted before accessioning. Kit and tube expiration dates and unique identifiers were verified, and patient and provider information was documented.

### Orthogonal Liquid Biopsy Comparison

A subset of the clinical samples were obtained via a prospective study from 2 cohorts to assess concordance with commercially available liquid biopsy CGP assays. A diverse cohort of patients, comprising individuals with different demographic backgrounds, medical histories, and clinical presentations, was enrolled in the study. The diversity of the sample cohorts, combined with the rigorous sample collection and processing procedures, ensured that the results obtained from this study are representative of a broad spectrum of clinical scenarios, facilitating the generalizability and clinical relevance of our findings.

Cohort 1 includes samples collected from patients at the same time that they were receiving a blood draw for a commercially available CGP test as part of their standard of care. Blood samples were collected from 182 patients from 6 community oncology practices across the United States. Inclusion criteria required stage III+ solid tumor, standard of care testing via Tempus xF or Tempus xF+, Guardant360CDx or Guardant360, Foundation One Liquid CDx, or Illumina TSO500 ctDNA, and informed consent.

Cohort 2 includes samples from a second set of patients. Blood samples were collected for the assay and for orthogonal testing at Intermountain Health Group during the same draw. Orthogonal test reports were generated using an established validated assay. Inclusion criteria required stage III+ solid tumor, current or planned targeted therapy. Patients were excluded from either cohort if they had received organ transplantation or non-autologous bone marrow or stem cell transplant, were pregnant, or were unable to provide consent.

Due to Northstar Select's higher sensitivity for multiple classes of alterations (SNV/Indel, CNV, etc), the proportion of patients with no pathogenic or clinically actionable alterations detected (negative report) was nearly half that of comparators (11% with no pathogenic alterations by Northstar Select vs 20% in comparators). Driven in large part by the additional detections below 0.5% VAF, Northstar Select measured a higher sensitivity for SNV/Indels than the comparators, with 51% more pathogenic and clinically actionable alterations combined.

### Quality Control and Metrics

As part of every run, sequencing metrics are monitored for issues, and a QC table is automatically generated as part of the bioinformatics pipeline. The following QC metrics were documented for all the sequencing batches done in the validation and all batches passed QC. The passing criteria is defined as sequencing error rate <1%, mean on-target-rate (OTR)>80%, and mean reads/sample >100M. Per sample QC pass criteria is determined by sample mean GE >600, sample OTR >60%, and sample sequencing depth >60M reads. In addition, hcQCTs are documented and used to assist troubleshooting. Sequencing error rate is the error rate of the sequencing run as calculated by Illumina. Mean GE is the mean genomic equivalents per sample, excluding QC fails. The mean OTR is the mean on target rate, or the percentage of aligned reads that were located on or near the baited (probe) regions.

The above validation data and analysis describes the results from the experimental arms performed to support the validation of the Northstar Select assay. The results show that the assay achieves high sensitivity, specificity, accuracy, and precision. By detecting clinically significant mutations in ctDNA in cancer patients, Northstar Select will enable oncologists to make well-informed decisions and treatment selections to improve clinical outcomes for their patients.

### Tumor Fraction Estimation and Tissue Copy Number Inference

Various embodiments of the disclosed method of tumor fraction (TF) estimation and tissue copy number (CN) inference use copy number signal from a liquid biopsy assay. In one embodiment, the method uses aneuploidy signal in cfDNA to estimate tumor fraction and infer tissue copy number, addressing a limitation of variant-based cfDNA assays, for benchmarking against gold-standard techniques like FISH.

In one embodiment, the treatment-selection liquid biopsy assay is employed for copy number quantification. The assay provides accurate plasma copy number measurement of genes and is resistant to technical and biological noise. A Gaussian mixture model (GMM) may be applied to quantify liquid aneuploidy signal on measured genes. The periodicity pattern can be used to estimate tumor fraction independently of SNV signal. In a validation study, 92 clinical samples (from 20 patients) from a databank of 3180 clinical samples were analyzed for concordance with SNV-based methods over multiple timepoints, and 136 clinical samples (from 128 patients) were identified with homozygous copy number loss calls for concordance with the tissue copy number estimate.

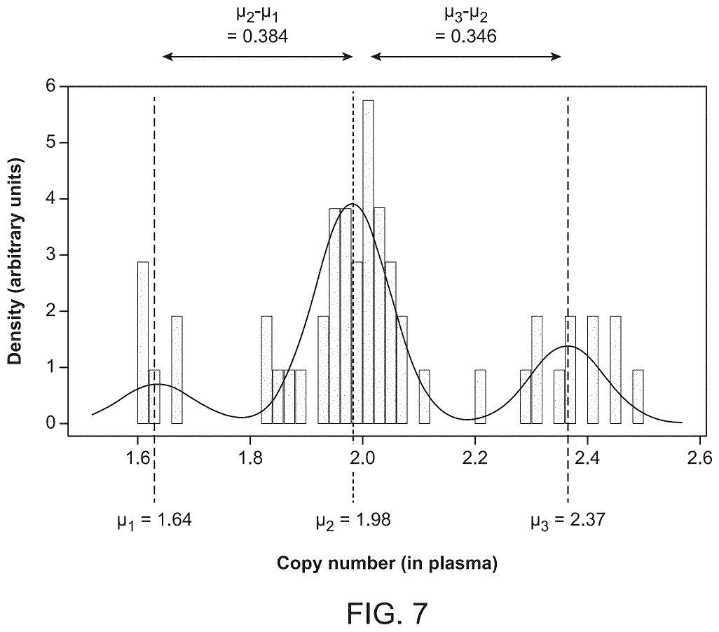

FIG. 7 shows a histogram of aneuploidy/copy number data in plasma-extracted cfDNA. The smooth curve with three peaks is the Gaussian mixture model (GMM) fit (with variances forced to be equal). The dotted line and dashed lines are the means of the fitted mixture components (μ1 and μ3 being a copy number loss or gain of 1, respectively, and μ2 being the diploid CN=2 call). The distances between the fitted u values are equal to the tumor fraction.

### Algorithm Principle and Application

In some embodiments, the CNV period calling algorithm is based on the principle of relating plasma copy number period to tumor fraction of cfDNA. In some embodiments, the following equation, stated from first principles:

$${CN}_{plasma} = TF \cdot {CN}_{tissue} + ( 1 - TF ) \cdot 2$$

can be rearranged to:

$$TF = \frac{{CN}_{plasma} - 2}{{CN}_{tissue} - 2}$$

The distribution of copy number in the plasma can be divided or classified into individual observations into hypothesized, discretized CNs in the tissue.

In some embodiments, the number outputted by the model fit, estimated_cnv_period, is the estimated distance between called peaks in the Gaussian mixture model fit to the data. In one embodiment, this value should be able to work in either direction, for instance, +1*estimated_cnv_period for a CN=3 in the tumor tissue, or in the down direction−1*estimated_cnv_period.

In some embodiments, this algorithm first tests the fits of mixture models with between 3 and 10 mixture components and gathers their Bayesian Information Criterion (BICs). This algorithm then computes the mixture component number penalty part of the BIC formula and uses it to calculate an “adjusted” BIC, where the penalty from adding more mixture components to the model is partially mitigated by a BIC_adjustment_coefficient that is set empirically (and can be further adjusted, from 0 to 1). In one embodiment, this adjustment coefficient can effectively reduce the penalty of adding more mixture components, allowing a higher number to be effectively picked from the set of test models.

In some embodiments, the next step of the algorithm involves trimming down the set of mixture components, where mixture component rows that are within the model's variance of one another are merged. When merging, the Gaussian means (u) and proportions undergo a weighted averaging, and the number of merged peaks in one line item post merging is reported in the column n_peaks_in_group.

In some embodiments, the next step of the algorithm involves calling the copy number period (CN_period), which is the distance between two peaks where the in-tissue copy number differs by one. The CN_period measure takes the same units as the x-axis of the CN distribution (i.e., units of CN in the plasma). In some embodiments, this is done by calculating the row-wise difference in the u's of the fitted Gaussians; this set of deltas is filtered to only keep the deltas within +1 standard deviation of the mean of the deltas. Post-filtering, the median delta is chosen as the CN_period.

In some embodiments, the next step of the algorithm involves using the CN_period to calculate the likely copy number in tissue for each of the peaks. A linear subtraction term can be used to center the peak closest to 2 at 2, and it applied to all other peaks as well. Then distance from 2 is divided by the CN_period to arrive at an estimated copy number in the tissue. In some embodiments, an alternative to Bayesian Information Criterion (BIC) can be used to select the mixture model. In some embodiments, peaks whose means are separated by less than the trimmed variance of the data can be merged. In some embodiments, other statistically-derived distances are used to determine which rows to merge.

In some embodiments, it is possible that the aneuploidy limit of quantification (LoQ), when processed through the GMM pipeline, translates to a tumor fraction of about 0.005, or 0.5%. In some embodiments, the LoQ for copy number calls from the CNV pipeline itself (whose data is the input into this pipeline) is likely in a higher range (e.g., about 1.5%-1.6%).

In some embodiments, the output of the CN calling algorithm can be compared to the copy number losses identified by an assay (such as the one described previously). A cut-off of estimated in-tissue copy number of 1.00 or below can be used in the GMM data to call samples with evidence of homozygous loss. In some embodiments, this analysis may be restricted to accession IDs that are in common between the two sets. The analysis may also be restricted to genes for which the assay makes loss calls, such as: “ATM”, “BRCA1”, “BRCA2”, “PTEN”, or “CDKN2A,” etc. For instance, of the losses called in variantDB, the GMM model called 89% of them. Assuming variantDB is the gold standard, the sensitivity of this method is thus 89%. Furthermore, the GMM was 100% specific: no additional accessions had losses called by GMM for the assay's CN loss reporting gene set (the gene set being (“ATM”, “BRCA1”, “BRCA2”, “PTEN”, “CDKN2A”).

| is_TF_low | varDB_loss_call | GMM_loss_call | n |
|---|---|---|---|
| Low TF | Not called | Present | 1 |
| Okay | Present | Not called | 15 |
| Okay | Present | Present | 126 |

$$126 / ( 15 + 126 ) = 0.893617 ( \sim 89 \% ) .$$

As shown above, for the remaining ˜11%, the GMM model failed to identify enough peaks to make a copy number period call, and therefore no in-tissue CN estimate was obtained. In some embodiments, the CN calling algorithm may also identify additional losses outside of the reportable gene set of the assay.

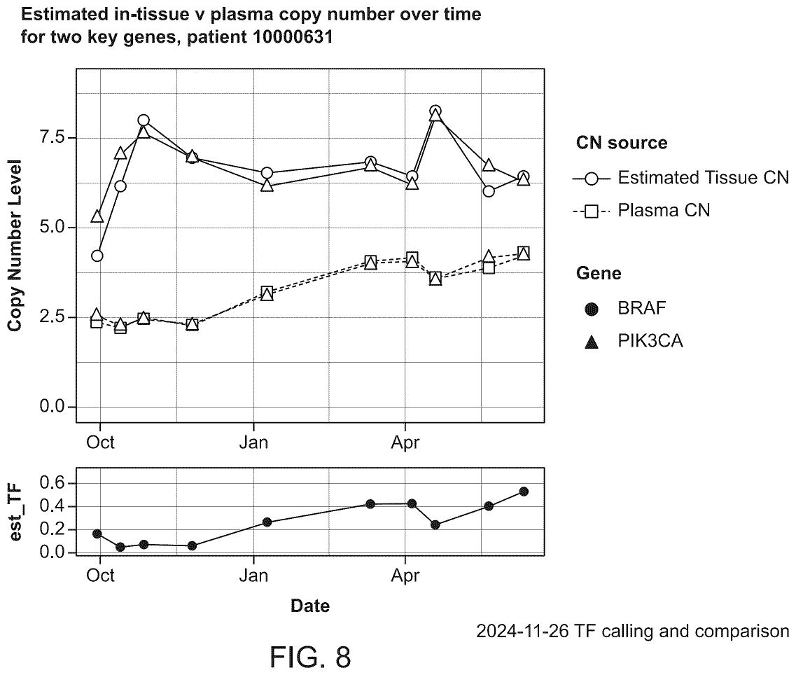

In some embodiments, data from the CN calling algorithm may be used to analyze a time series of one or more patients. FIG. 8 illustrates the estimated in-tissue vs plasma copy number (CN) over time for two key genes (BRAF and PIK3CA) for one patient. The hypothesis from another study (e.g., MolDx study) showed that PIK3CA has an in-tissue CN of ˜6. Analysis of serially tested clinical plasma samples revealed that—despite large tumor fraction fluctuations (≤5% to ≥50%)—tissue copy number estimates for the same patient remained stable. Note the top 2 variants (BRAF and PIK3CA) were called by the CNV pipeline as amplified across the different requisitions. Overall, this is consistent with previous observations and cancer biology that the tumor copy number remains the same regardless of tumor burden changes in the plasma samples.

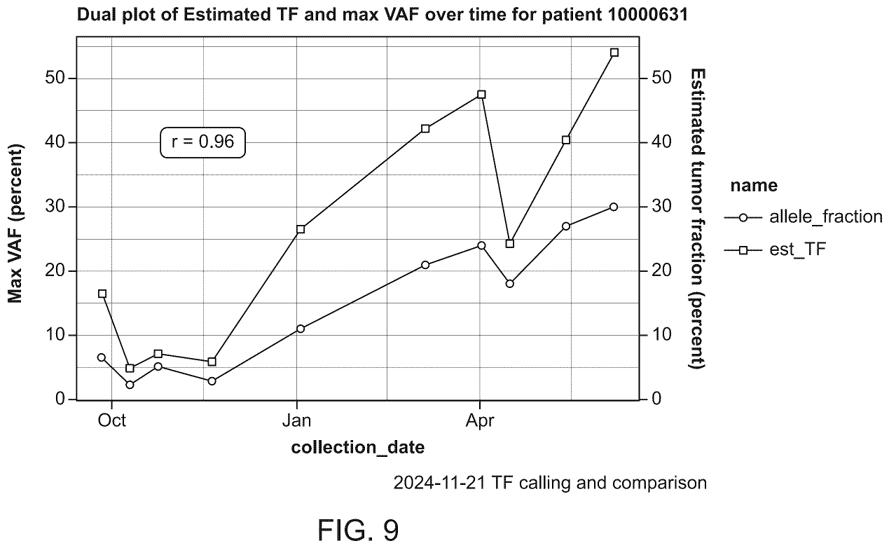

FIG. 9 illustrates estimated tumor fraction (TF) and max VAF over time for the above patient. As shown, the estimated TF method using CN calling can achieve a high correlation (e.g., r=0.96 in this case) with the VAF method in patients with longitudinally trackable SNVs. In some embodiments, for patients without trackable SNVs, a combinatorial approach may be used to improve the TF estimation. For instance, a number of signals or data sources, including but not limited to maximum/average SNV signal, Gaussian fitting to aneuploidy signal, and/or methylation signal, may be used in combination to achieve a better TF estimation. In some embodiments, each signal or data source may be attributed a weight value based on one or more metrics including the accuracy, consistency, or fidelity of data. In some embodiments, ground truth data may be collected for data calibration.

### APPENDIX A: LIST OF MUTATIONS IN CONTRIVED TUMOR SAMPLES

### List of Clinically Relevant Mutations, Expected and Detected VAF % s (or Copy Numbers) in 10% Contrived Tumor Samples

| | | | Expected | Detected by | Detected VAF % (or CN) | |
|---|---|---|---|---|---|---|
| | | Sample | VAF % | Northstar Select in | | Rep. 2 |
| Mutation | Type | ID | (or CN) | all rep. performed | Rep. 1 | (if run) |
| APC | SNV | 14-502 | 2.9% | Yes | 5.85 | 4.7 |
| Y935* | | | | | | |
| ERBB2 | amplification | 14-502 | 3.62 copies | Yes | 4.95 copies | 4.67 copies |
| EGFR | SNV | 101211 | 5.2% | Yes | 9.19 | |
| L858R | | | | | | |
| EGFR | SNV | 101211 | 1.1% | Yes | 2.59 | |
| T790M | | | | | | |
| RB1 | SNV | 101211 | 2.3% | Yes | 2.84 | |
| S751C | | | | | | |
| PTEN | Insertion | 101211 | 5.0% | Yes | 3.54 | |
| V191fs*6 | | | | | | |
| PIK3CA | SNV | 17-090 | 2.60% | Yes | 2.04 | |
| E545K | | | | | | |
| PTEN | SNV | 17-090 | 4.90% | Yes | 3.3 | |
| Q214* | | | | | | |
| ARID1A | SNV | 17-090 | 2.10% | Yes | 1.42 | |
| Q611* | | | | | | |
| CDH1 | Deletion | 17-090 | 4.90% | Yes | 3.08 | |
| Q307fs*45 | | | | | | |
| TP53 | SNV | 17-833 | 7.40% | Yes | 5.24 | 4.65 |
| E204* | | | | | | |
| STK11 | Deletion | 17-833 | 7.40% | Yes | 5.74 | 5.9 |
| L282fs*5 | | | | | | |
| KRAS | amplification | 17-833 | 4.40 copies | Yes | 4.02 copies | 4.08 copies |
| MYC | amplification | 17-833 | 2.45 copies | Yes | 2.36 copies | 2.26 copies |
| KRAS | SNV | 20-0152 | 1.70% | Yes | 1.66 | |
| G12C | | | | | | |
| TP53 | SNV | 20-0152 | 3.20% | Yes | 3.22 | |
| R249S | | | | | | |
| ARID1A | SNV | 20-0152 | 1.20% | Yes | 0.96 | |
| P559fs*60 | | | | | | |
| BRCA1 | SNV | 20-0152 | 1.20% | Yes | 1.08 | |
| E1358Q | | | | | | |
| TERT | SNV | 20-0152 | 2.20% | Yes | 2.04 | |
| promoter | | | | | | |
| c.-124C > T | | | | | | |
| KRAS | SNV | 102045 | 15.77% | Yes | 6.2 | 6.44 |
| G12D | | | | | | |
| TP53 | SNV | 102045 | 1.18% | Yes | 0.97 | 1.62 |
| R213* | | | | | | |
| JAK2 | SNV | 102045 | 0.59% | Yes | 0.94 | 1.12 |
| V617F | | | | | | |
| CDK4 | amplification | 102045 | 2.58 copies | Yes | 2.42 copies | 2.52 copies |
| TP53 | SNV | 15-780 | 1.71% | Yes | 2.31 | |
| M237I | | | | | | |
| TP53 | Insertion | 15-780 | 2.54% | Yes | 2.34 | |
| V272fs*34 | | | | | | |
| TP53 | SNV | 15-780 | 1.82% | Yes | 1.28 | |
| R248L | | | | | | |
| MET | SNV | 15-780 | 1.60% | Yes | 1.1 | |
| L1195F | | | | | | |
| BRIP1 | SNV | 15-780 | 0.82%1 | Yes | 0.82 | |
| Q553* | | | | | | |
| PTEN | SNV | 17-1110 | 3.14% | Yes | 4.57 | 6.67 |
| E284* | | | | | | |
| TP53 | SNV | 17-1110 | 5.35% | Yes | 5.72 | 5.19 |
| H179R | | | | | | |
| NF1 | Deletion | 17-1110 | 4.83% | Yes | 4.83 | 4.96 |
| G444fs*29 | | | | | | |
| PIK3CA | SNV | 17-1110 | 3.70% | Yes | 3.71 | 4.47 |
| E545K | | | | | | |
| RET | amplification | 17-1110 | 2.71 copies | Yes | 2.70 copies | 2.71 copies |
| TP53 | SNV | 102372 | 3.82% | Yes | 4.71 | 4.69 |
| Y107* | | | | | | |
| STK11 | Indel/ | 102372 | 14.67% | Yes | 5.18 | 5.03 |
| S193_D1 | Complex | | | | | |
| 94delinsFY | | | | | | |
| CDK4 | amplification | 102372 | 3.85 copies | Yes | 3.78 copies | 3.90 copies |
| MYC | amplification | 102372 | 2.46 copies | Yes | 2.38 copies | 2.34 copies |
| TERT | SNV | 18-0586 | 2.83% | Yes | 3.74 | |
| promoter | | | | | | |
| c.-146C > T | | | | | | |
| APC | SNV | 13-533 | 1.95% | Yes | 2.09% | |
| L563* | | | | | | |
| APC | SNV | 13-533 | 3.96% | Yes | 3.98% | |
| Q1367* | | | | | | |
| TP53 | SNV | 13-533 | 4.42% | Yes | 7.24% | |
| S241Y | | | | | | |
| CTNNBI | SNV | 14-0231 | 0.93% | Yes | 2.02% | |
| W383R | | | | | | |
| KRAS | SNV | 14-0231 | 3.33% | Yes | 4.85% | |
| G13D | | | | | | |
| APC | SNV | 14-613 | 4.94% | Yes | 3.67% | |
| R302* | | | | | | |
| TP53 | SNV | 14-715 | 0.50% | Yes | 0.95% | |
| P278R | | | | | | |
| PIK3CA | SNV | 17-617 | 2.86% | Yes | 2.68% | |
| H1047R | | | | | | |
| TP53 | SNV | 17-617 | 2.90% | Yes | 2.06% | |
| R196* | | | | | | |
| KIT | amplification | 17-617 | 2.452 copies | Yes | 2.30 copies in | |
| | | | | | anp range | |
| AR | amplification | 17-617 | 3.61 copies | Yes | 3.55 copies | |
| CDKN2A | Insertion | 18-1066 | 1.64% | Yes | 1.16% | |
| Y44fs*76 | | | | | | |
| KRAS | SNV | 18-1066 | 2.40% | Yes | 2.54% | |
| G12V | | | | | | |
| ARIDA1A | SNV | 19-0064 | 2.90% | Yes | 2.82% | |
| Q611* | | | | | | |
| ARIDA1A | Deletion | 19-0064 | 0.26% | Yes | 0.18% | |
| S2149fs*51 | | | | | | |
| PIK3CA | SNV | 19-0064 | 2.42% | Yes | 3.60% | |
| E545K | | | | | | |
| TP53 | SNV | 19-0064 | 2.50% | Yes | 3.04% | |
| R248Q | | | | | | |
| TP53 | SNV | 19-0064 | 1.28% | Yes | 1.84% | |
| C238Y | | | | | | |
| TP53 | SNV | 19-0064 | 0.55% | Yes | 0.37% | |
| S215G | | | | | | |
| NF1 | SNV | 19-0064 | 3.43% | Yes | 3.90% | |
| E318* | | | | | | |
| NF1 | SNV | 19-0064 | 0.51% | Yes | 0.12% | |
| Q2373* | | | | | | |
| CHEK2 | SNV | 19-0064 | 1.21% | Yes | 0.90% | |
| R346C | | | | | | |
| CHEK2 | SNV | 19-0064 | 0.76% | Yes | 0.57% | |
| I160T | | | | | | |
| CHEK2 | SNV | 19-0064 | 0.77% | Yes | 0.55% | |
| R137* | | | | | | |
| CHEK2 | SNV | 19-0064 | 0.66% | Yes | 0.81% | |
| 319 + | (splicing) | | | | | |
| 1G > A | | | | | | |
| PTEN | loss | 19-0064 | 1.67 copies | Yes | 1.82 copies | |
| FGFR1 | SNV | 107063 | 0.60% | Yes | 1.15% | |
| P283L | | | | | | |
| PTEN | Deletion | 107063 | 2.82% | Yes | 2.45% | |
| N329fs*14 | | | | | | |
| BRCA1 | Indel/ | 107063 | 3.38% | Yes | 3.39% | |
| K795_T900delins | Complex | | | | | |
| NILLQIP | | | | | | |
| EKECFK | | | | | | |
| PDLSKE | | | | | | |
| VSTLG | | | | | | |

¹ This is a confirmed mutation but TSO500 had a false negative: low DP, low support
² KIT copy number was confirmed at pure tumor <6 copies, hence calling aneuploidy level is acceptable

### List of MSI Tumors and Diluted VAF Samples

| | | Called MSI-H by Northstar |
|---|---|---|
| | Orthogonal | Select in all rep. Performed |
| Sample ID | MSI Call | (# of Replicates) |
| 21-0189 | MSI-H | Yes (1) |
| 19-0316 | MSI-H | Yes (1) |
| 14-565 | MSI-H | Yes (1) |
| 14-267 | MSI-H | Yes (1) |
| 14-196 | MSI-H | Yes (1) |
| 16-831 | MSI-H | Yes (1) |
| 15-004 | MSI-H | Yes (1) |
| 18-0978 | MSI-H | Yes (1) |
| 21-189_1% | MSI-H | Yes (4) |
| 21-189_0.5% | MSI-H | Yes (2) |
| 21-189_0.25% | MSI-H | Yes (2) |
| 19-0316_1% | MSI-H | Yes (2) |
| 19-0316_0.5% | MSI-H | Yes (2) |
| 19-0316_0.25% | MSI-H | Yes (2) |
| 14-565_1% | MSI-H | Yes (2) |
| 14-565_0.5% | MSI-H | Yes (1) |
| 14-565_0.25% | MSI-H | Yes (2) |

### APPENDIX B: MUTATIONS TESTED IN TWIST PAN CANCER REFERENCE MATERIAL

### List of 279 Small Variants in 3rd Party Synthetic Material

| | Mutation | | Chrom | Pos | Reference | Alternate | |
|---|---|---|---|---|---|---|---|
| Gene | description | Mutation ID | (hg19) | (hg19) | sequence | sequence | Variant category |
| ARID1A | Q1401* | COSM51417 | chr1 | 27100919 | C | T | single_base_substitution |
| ARID1A | M1564Hfs*8 | COSM211769 | chr1 | 27101401 | G | GC | short_insertion |
| MPL | W515L | COSM18918 | chr1 | 43815009 | G | T | single_base_substitution |
| NRAS | Q61H | COSM586 | chr1 | 115256528 | T | G | single_base_substitution |
| NRAS | G12D | COSM564 | chr1 | 115258747 | C | T | single_base_substitution |
| RIT1 | M90I | COSM357927 | chr1 | 155874261 | C | T | single_base_substitution |
| DDR2 | I638F | COSM7363943 | chr1 | 162745497 | A | T | single_base_substitution |
| ALK | P1543S | COSM2941442 | chr2 | 29416326 | G | A | single_base_substitution |
| ALK | R1275Q | COSM28056 | chr2 | 29432664 | C | T | single_base_substitution |
| ALK | F1174L | COSM28055 | chr2 | 29443695 | G | T | single_base_substitution |
| ALK | G1128A | COSM98475 | chr2 | 29445450 | C | G | single_base_substitution |
| IDH1 | R132C | COSM28747 | chr2 | 209113113 | G | A | single_base_substitution |
| ALK | | COSM7408659 | chr2 | 29419689 | C | T | single_base_substitution |
| ALK | | COSM6924954 | chr2 | 29420441 | C | T | single_base_substitution |
| ALK | | COSM6939221 | chr2 | 29430047 | T | C | single_base_substitution |
| ALK | | COSM28617 | chr2 | 129436893 | C | T | single_base_substitution |
| ALK | | COSM6949625 | chr2 | 29446310 | G | A | single_base_substitution |
| VHL | F148Lfs*11 | COSM14410 | chr3 | 10188296 | AT | A | short_deletion |
| VHL | R161* | COSM17612 | chr3 | 10191488 | C | T | single_base_substitution |
| MLH1 | R498fs | COSM5895322 | chr3 | 37070354 | CG | C | short_deletion |
| CTNNB1 | T41A | COSM5664 | chr3 | 41266124 | A | G | single_base_substitution |
| RHOA | Y42C | COSM2849892 | chr3 | 49412898 | T | C | single_base_substitution |
| PIK3CA | G106_R108del | COSM13475 | chr3 | 178916928 | AGGCAACCGT | A | long_deletion |
| PIK3CA | N345K | COSM754 | chr3 | 178921553 | T | A | single_base_substitution |
| PIK3CA | E545K | COSM763 | chr3 | 178936091 | G | A | single_base_substitution |
| PIK3CA | H1047R | COSM775 | chr3 | 178952085 | A | G | single_base_substitution |
| CTNNB1 | | COSM6845286 | chr3 | 41266554 | TCATCCCA | T | long_deletion |
| CTNNB1 | | CTNNB1_exon_ | chr3 | 41267223 | TAAAATGGCA | A | complex |
| | | 7_indel | | | GT | | |
| CTNNB1 | | COSM5608170 | chr3 | 41268765 | AAACT | A | medium_deletion |
| CTNNB1 | | COSM6853630 | chr3 | 41274898 | G | GGGA | medium_insertion |
| PIK3CA | | COSM3205605 | chr3 | 178917610 | G | A | single_base_substitution |
| PIK3CA | | COSM6931303 | chr3 | 178919264 | A | T | single_base_substitution |
| PIK3CA | | COSM21450 | chr3 | 178922364 | G | T | single_base_substitution |
| PIK3CA | | COSM5613085 | chr3 | 178927462 | GT | G | short_deletion |
| PIK3CA | | COSM6940128 | chr3 | 178928078 | AGAAGATTTG | A | long_deletion |
| | | | | | CTGAACCCTA | | |
| | | | | | TTGGTGTTAC | | |
| | | | | | T | | |
| PIK3CA | | COSM1716809 | chr3 | 178937016 | C | T | single_base_substitution |
| PIK3CA | | COSM5751700 | chr3 | 178937370 | G | GT | short_insertion |
| PIK3CA | | COSM250052 | chr3 | 178937738 | T | C | single_base_substitution |
| PIK3CA | | COSM6911769 | chr3 | 178938922 | GAGA | G | medium_deletion |
| PIK3CA | | COSM6475729 | chr3 | 178941911 | T | C | single_base_substitution |
| PIK3CA | | COSM6981846 | chr3 | 178942528 | C | A | single_base_substitution |
| PIK3CA | | COSM1041507 | chr3 | 178943785 | C | T | single_base_substitution |
| PIK3CA | | COSM39499 | chr3 | 178947162 | G | C | single_base_substitution |
| PIK3CA | | COSM769 | chr3 | 178947827 | G | T | single_base_substitution |
| PIK3CA | | COSM6475740 | chr3 | 178948161 | A | G | single_base_substitution |
| FGFR3 | S249C | COSM715 | chr4 | 1803568 | C | G | single_base_substitution |
| FGFR3 | Y375C | COSM718 | chr4 | 1806099 | A | G | single_base_substitution |
| FGFR3 | K652E | COSM719 | chr4 | 1807889 | A | G | single_base_substitution |
| PDGFRA | S566_ | COSM30546 | chr4 | 55141051 | GCCCAGATGG | G | long_deletion |
| | E571delinsR | | | | ACATGA | | |
| PDGFRA | N659K | COSM22414 | chr4 | 155144148 | C | G | single_base_substitution |
| PDGFRA | D842V | COSM736 | chr4 | 55152093 | A | T | single_base_substitution |
| KIT | L576P | COSM1290 | chr4 | 55593661 | T | C | single_base_substitution |
| KIT | K642E | COSM1304 | chr4 | 55594221 | A | G | single_base_substitution |
| KIT | D816V | COSM1314 | chr4 | 55599321 | A | T | single_base_substitution |
| FBXW7 | R465C | COSM22932 | chr4 | 153249385 | G | A | single_base_substitution |
| FGFR3 | | COSM7448276 | chr4 | 1808295 | T | TTGGGAGAT | long_insertion |
| | | | | | | CTTGCAC | |
| FGFR3 | | FGFR3_exon_ | chr4 | 1808948 | C | CGA | medium_insertion |
| | | 18_indel | | | | | |
| PDGFRA | | COSM6958142 | chr4 | 55144589 | A | C | single_base_substitution |
| KIT | | COSM6951399 | chr4 | 55524240 | TC | T | short_deletion |
| KIT | | COSM7345631 | chr4 | 55561938 | TTTG | T | medium_deletion |
| KIT | | COSM7345632 | chr4 | 55564681 | AG | A | short_deletion |
| KIT | | COSM6909371 | chr4 | 55565852 | G | T | single_base_substitution |
| KIT | | COSM3301432 | chr4 | 55569926 | G | A | single_base_substitution |
| KIT | | COSM9500507 | chr4 | 55573299 | A | G | single_base_substitution |
| KIT | | COSM6005552 | chr4 | 55575593 | C | T | single_base_substitution |
| KIT | | COSM6008883 | chr4 | 55589771 | A | ACGATTTT | long_insertion |
| KIT | | COSM53306 | chr4 | 55592178 | C | CTGCCTT | long_insertion |
| KIT | | COSM1305 | chr4 | 55595617 | TATAAGA | T | long_deletion |
| KIT | | COSM1306 | chr4 | 55597490 | CCAG | C | medium_deletion |
| KIT | | COSM28578 | chr4 | 55598133 | CA | C | short_deletion |
| KIT | | KIT_exon_18_ | chr4 | 55602765 | G | A | single_base_substitution |
| | | substitution | | | | | |
| KIT | | COSM6945539 | chr4 | 55603391 | C | A | single_base_substitution |
| KIT | | COSM6965292 | chr4 | 55604631 | CAG | C | medium_deletion |
| TERT | C228G | tert_c228g | chr5 | 1295344 | G | A | single_base_substitution |
| APC | R213* | COSM13134 | chr5 | 112116592 | C | T | single_base_substitution |
| APC | A1002Gfs*6 | COSM5748894 | chr5 | 112174294 | A | AG | short_insertion |
| APC | E1309Dfs*4 | COSM13113 | chr5 | 112175211 | TAAAAG | T | medium_deletion |
| APC | R1450* | COSM13127 | chr5 | 112175639 | C | T | single_base_substitution |
| APC | R2714C | COSM2991126 | chr5 | 112179431 | C | T | single_base_substitution |
| NPM1 | W288fs*12 | COSM17559 | chr5 | 170837543 | C | CTCTG | medium_insertion |
| APC | | COSM6963650 | chr5 | 112090720 | AAGGTATC | A | long_deletion |
| APC | | COSM6853815 | chr5 | 112102087 | AT | A | short_deletion |
| APC | | COSM9113053 | chr5 | 112103077 | G | GGAGAAAG | long_insertion |
| | | | | | | A | |
| APC | | COSM6854236 | chr5 | 112111407 | AATAG | A | medium_deletion |
| APC | | COSM6976104 | chr5 | 112128165 | AAATCG | A | medium_deletion |
| APC | | COSM6984704 | chr5 | 112136981 | ATC | A | medium_deletion |
| APC | | COSM5010340 | chr5 | 112151290 | G | GATGTTT | long_insertion |
| APC | | COSM25155 | chr5 | 112154871 | C | CG | short_insertion |
| APC | | COSM6971752 | chr5 | 112157639 | AATGAAACTT | A | long_deletion |
| | | | | | TCATTTG | | |
| APC | | COSM4169285 | chr5 | 112162818 | CATTGCAGAA | C | long_deletion |
| | | | | | TT | | |
| APC | | COSM1169625 | chr5 | 112163634 | ATGCTC | A | medium_deletion |
| APC | | COSM4169180 | chr5 | 112164560 | CGAGT | C | medium_deletion |
| APC | | COSM6854200 | chr5 | 112170838 | T | TA | short_insertion |
| ESR1 | D538G | COSM94250 | chr6 | 152419926 | A | G | single_base_substitution |
| ROS1 | | COSM9513198 | chr6 | 117642554 | C | A | single_base_substitution |
| ROS1 | | COSM6941244 | chr6 | 117645500 | G | A | single_base_substitution |
| ROS1 | | COSM6959063 | chr6 | 117647457 | TCTGAA | T | medium_deletion |
| ROS1 | | COSM6965416 | chr6 | 117650522 | C | A | single_base_substitution |
| ROS1 | | COSM9125580 | chr6 | 117658490 | T | C | single_base_substitution |
| EGFR | L718Q | COSM6503269 | chr7 | 55241705 | T | A | single_base_substitution |
| EGFR | E746_ | COSM6225 | chr7 | 55242465 | GGAATTAAGA | G | long_deletion |
| | A750delELREA | | | | GAAGCA | | |
| EGFR | T790M | COSM6240 | chr7 | 55249071 | C | T | single_base_substitution |
| EGFR | L858R | COSM6224 | chr7 | 55259515 | T | G | single_base_substitution |
| MET | d1246n | COSM5015794 | chr7 | 116423407 | G | A | single_base_substitution |
| SMO | D473H | COSM34198 | chr7 | 128849189 | G | C | single_base_substitution |
| BRAF | p.V600E | COSM476 | chr7 | 140453136 | A | T | single_base_substitution |
| EZH2 | Y641F | COSM37028 | chr7 | 48508727 | T | A | single_base_substitution |
| EGFR | | COSM6937748 | chr7 | 55087046 | G | A | single_base_substitution |
| EGFR | | EGFR_exon_2_ | chr7 | 55209986 | AGGCACG | C | complex |
| | | indel | | | | | |
| EGFR | | COSM9233361 | chr7 | 55211109 | G | T | single_base_substitution |
| EGFR | | EGFR_exon_4_ | chr7 | 55214348 | C | A | single_base_substitution |
| | | substitution | | | | | |
| EGFR | | COSM7002280 | chr7 | 55219001 | C | G | single_base_substitution |
| EGFR | | COSM4166393 | chr7 | 55220320 | C | T | single_base_substitution |
| EGFR | | COSM9494233 | chr7 | 55221796 | CCCCGAGGG | C | long_deletion |
| EGFR | | COSM6962235 | chr7 | 55223530 | TGTG | T | medium_deletion |
| EGFR | | COSM6970489 | chr7 | 55224495 | G | A | single_base_substitution |
| EGFR | | COSM7002279 | chr7 | 55225428 | G | A | single_base_substitution |
| EGFR | | COSM236670 | chr7 | 55228009 | C | A | single_base_substitution |
| EGFR | | COSM7343128 | chr7 | 55229231 | AG | A | short_deletion |
| EGFR | | COSM5530405 | chr7 | 55231427 | G | A | single_base_substitution |
| EGFR | | COSM6196864 | chr7 | 55233067 | TG | T | short_deletion |
| EGFR | | EGFR_exon_16_ | chr7 | 55238874 | T | C | single_base_substitution |
| | | substitution | | | | | |
| EGFR | | COSM6909028 | chr7 | 55240741 | TG | T | short_deletion |
| EGFR | | COSM6976991 | chr7 | 55260477 | G | A | single_base_substitution |
| EGFR | | COSM6932208 | chr7 | 55266482 | C | T | single_base_substitution |
| EGFR | | COSM5762244 | chr7 | 55268044 | C | T | single_base_substitution |
| EGFR | | COSM3762773 | chr7 | 55268916 | C | T | single_base_substitution |
| EGFR | | COSM6925302 | chr7 | 55269458 | T | C | single_base_substitution |
| EGFR | | COSM7410173 | chr7 | 55270220 | G | A | single_base_substitution |
| EGFR | | COSM9496259 | chr7 | 55273218 | G | A | single_base_substitution |
| MET | | COSM6912457 | chr7 | 116339746 | CTTCT | C | medium_deletion |
| MET | | COSM5945634 | chr7 | 116371771 | G | A | single_base_substitution |
| MET | | COSM6927005 | chr7 | 116380020 | C | T | single_base_substitution |
| MET | | COSM6975700 | chr7 | 116380943 | ATTTCCAGTC | A | long_deletion |
| | | | | | CTGCAG | | |
| MET | | MET_exon_6_ | chr7 | 116395509 | CTAGAGTTCT | CG | complex |
| | | indel | | | CCTTGGAAAT | | |
| | | | | | GAGAG | | |
| MET | | COSM3632213 | chr7 | 116397781 | G | A | single_base_substitution |
| MET | | COSM5977594 | chr7 | 116398541 | AC | A | short_deletion |
| MET | | COSM6937367 | chr7 | 116399448 | TG | T | short_deletion |
| MET | | COSM6957131 | chr7 | 116403181 | C | CA | short_insertion |
| MET | | COSM5047343 | chr7 | 116409843 | G | C | single_base_substitution |
| MET | | COSM5609378 | chr7 | 116411678 | C | T | single_base_substitution |
| MET | | COSM6984036 | chr7 | 116414994 | GACATGTCCC | G | long_deletion |
| | | | | | CCA | | |
| MET | Exon14 skip | MET_exon_14_ | chr7 | 116412044 | G | A | single_base_substitution |
| | | skip | | | | | |
| MET | | COSM6438054 | chr7 | 116417494 | A | T | single_base_substitution |
| MET | | MET_exon_17_ | chr7 | 116418865 | A | ATT | medium_insertion |
| | | indel | | | | | |
| MET | | COSM1579075 | chr7 | 116422110 | CA | C | short_deletion |
| MET | | COSM7345743 | chr7 | 116436056 | CATGTGAACG | C | long_deletion |
| | | | | | CTACTT | | |
| FGFR1 | K656E | COSM35673 | chr8 | 38272308 | T | C | single_base_substitution |
| FGFR1 | N546K | COSM19176 | chr8 | 38274849 | G | T | single_base_substitution |
| JAK2 | V617F | COSM12600 | chr9 | 5073770 | G | T | single_base_substitution |
| GNAQ | Q209P | COSM28758 | chr9 | 80409488 | T | G | single_base_substitution |
| NOTCH1 | P2514Rfs*4 | COSM12774 | chr9 | 139390648 | CAG | C | medium_deletion |
| NOTCH1 | L1600Pfs*10 | COSM5751249 | chr9 | 139399344 | A | AG | short_insertion |
| GATA3 | P408fs | COSM166059 | chr10 | 18115874 | C | CG | short_insertion |
| RET | M918T | COSM965 | chr10 | 43617416 | T | C | single_base_substitution |
| PTEN | R130G | COSM5033 | chr10 | 89692905 | G | A | single_base_substitution |
| PTEN | D268Gfs*30 | COSM5012 | chr10 | 89717769 | T | TA | short_insertion |
| PTEN | N323Kfs*2 | COSM4990 | chr10 | 89720811 | C | CA | short_insertion |
| FGFR2 | K659E | COSM36909 | chr10 | 123247516 | T | C | single_base_substitution |
| FGFR2 | N549K | COSM36912 | chr10 | 123258034 | A | T | single_base_substitution |
| FGFR2 | C382R | COSM36906 | chr10 | 123274774 | A | G | single_base_substitution |
| FGFR2 | S252W | COSM36903 | chr10 | 123279677 | G | C | single_base_substitution |
| RET | | COSM984 | chr10 | 43609073 | ACTGCTTCCC | A | long_deletion |
| | | | | | TGAGGAGGAG | | |
| | | | | | AAGTGCTT | | |
| RET | | RET_exon_13_ | chr10 | 43613843 | G | A | single_base_substitution |
| | | substitution | | | | | |
| RET | | COSM6945831 | chr10 | 43615072 | G | T | single_base_substitution |
| RET | | COSM962 | chr10 | 43615611 | GAGATGTTTA | G | long_deletion |
| | | | | | TGA | | |
| PTEN | | COSM7350864 | chr10 | 189624293 | T | TCGGGAGC | long_insertion |
| PTEN | | COSM5346960 | chr10 | 189653813 | T | TATGGGATT | long_insertion |
| | | | | | | G | |
| PTEN | | COSM5882 | chr10 | 189685306 | A | ATAT | medium_insertion |
| PTEN | | COSM1173605 | chr10 | 89690826 | C | CTA | medium_insertion |
| PTEN | | COSM7347202 | chr10 | 89711937 | T | TCACCGA | long_insertion |
| HRAS | G12V | COSM483 | chr11 | 534288 | C | A | single_base_substitution |
| CCND1 | T286I | COSM931395 | chr11 | 169466019 | C | T | single_base_substitution |
| ATM | S214Pfs*16 | COSM1350740 | chr11 | 108114816 | CT | C | short_deletion |
| ATM | | COSM1315819 | chr11 | 108098351 | CATGAGTCTA | C | long_deletion |
| | | | | | GTACTTAATG | | |
| ATM | | COSM6978979 | chr11 | 108100042 | CAAACAGAA | C | long_deletion |
| ATM | | COSM758337 | chr11 | 108106541 | TATCTC | T | medium_deletion |
| ATM | | COSM3733253 | chr11 | 108115679 | AAG | A | medium_deletion |
| ATM | | COSM1235448 | chr11 | 108117789 | T | TA | short_insertion |
| ATM | | COSM1235427 | chr11 | 108119772 | GGGAAGTA | G | long_deletion |
| ATM | | ATM_exon_10_ | chr11 | 108121455 | AAA | T | complex |
| | | indel | | | | | |
| ATM | | COSM6935895 | chr11 | 108122672 | AAAGGAATC | A | long_deletion |
| ATM | | COSM6958308 | chr11 | 108123565 | GGA | G | medium_deletion |
| ATM | | ATM_exon_13_ | chr11 | 108124719 | TGTCTTCTGG | G | complex |
| | | indel | | | GATTATCAGA | | |
| | | | | | A | | |
| ATM | | ATM_exon_14_ | chr11 | 108127025 | T | TAAAAA | medium_insertion |
| | | indel | | | | | |
| ATM | | COSM6986880 | chr11 | 108128325 | TGTACCA | T | long_deletion |
| ATM | | COSM9179264 | chr11 | 108129792 | GTAAAAGTTT | G | long_deletion |
| | | | | | AGTAAGTA | | |
| ATM | | COSM6856770 | chr11 | 108138061 | GTACCA | G | medium_deletion |
| ATM | | COSM6906886 | chr11 | 108139290 | TTGATTCTAG | T | long_deletion |
| | | | | | CACGC | | |
| ATM | | COSM7345428 | chr11 | 108142010 | ATGTT | A | medium_deletion |
| ATM | | COSM7345432 | chr11 | 108143488 | GGA | G | medium_deletion |
| ATM | | COSM1235411 | chr11 | 108150246 | TC | T | short_deletion |
| ATM | | COSM9493731 | chr11 | 108151889 | GA | G | short_deletion |
| ATM | | COSM758341 | chr11 | 108153579 | ACTACACAAA | A | long_deletion |
| | | | | | TATTGAGG | | |
| ATM | | COSM21638 | chr11 | 108155116 | CAGAGACA | C | long_deletion |
| ATM | | COSM6958310 | chr11 | 108158371 | GTTA | G | medium_deletion |
| ATM | | COSM6971320 | chr11 | 108159737 | TC | T | short_deletion |
| ATM | | COSM6956709 | chr11 | 108160422 | CTGTT | C | medium_deletion |
| ATM | | COSM9312366 | chr11 | 108163485 | C | CATAA | medium_insertion |
| ATM | | COSM9358682 | chr11 | 108164066 | G | GGATA | medium_insertion |
| ATM | | ATM_exon_32_ | chr11 | 108165711 | GAAGGACTAA | T | complex |
| | | indel | | | AGGATCTTCG | | |
| | | | | | AAGA | | |
| ATM | | COSM4745906 | chr11 | 108168096 | AAAAG | A | medium_deletion |
| ATM | | COSM22533 | chr11 | 108170481 | TTTCTC | T | medium_deletion |
| ATM | | COSM22526 | chr11 | 108172397 | GTTACCTGT | G | long_deletion |
| ATM | | COSM7347299 | chr11 | 108173584 | TAGA | T | medium_deletion |
| ATM | | COSM9358193 | chr11 | 108175412 | AC | A | short_deletion |
| ATM | | COSM1315822 | chr11 | 108178696 | TGAG | T | medium_deletion |
| ATM | | COSM5967541 | chr11 | 108181013 | TAAGAAAAGT | T | long_deletion |
| | | | | | ATGGATGATC | | |
| | | | | | AAG | | |
| ATM | | COSM21325 | chr11 | 108183192 | A | C | single_base_substitution |
| ATM | | COSM1235422 | chr11 | 108186787 | TA | T | short_deletion |
| ATM | | ATM_exon_43_ | chr11 | 108188188 | AAGAA | TC | complex |
| | | indel | | | | | |
| ATM | | COSM6911065 | chr11 | 108190685 | GA | G | short_deletion |
| ATM | | COSM7343670 | chr11 | 108192057 | G | A | single_base_substitution |
| ATM | | COSM21644 | chr11 | 108196170 | GAA | G | medium_deletion |
| ATM | | COSM6933908 | chr11 | 108196879 | CA | C | short_deletion |
| ATM | | COSM758343 | chr11 | 108198384 | CTAAAACT | C | long_deletion |
| ATM | | COSM6977654 | chr11 | 108199755 | AAG | A | medium_deletion |
| ATM | | COSM6986181 | chr11 | 108200942 | TACAC | T | medium_deletion |
| ATM | | COSM6853938 | chr11 | 108202261 | G | GA | short_insertion |
| ATM | | COSM6936524 | chr11 | 108202687 | T | TA | short_insertion |
| ATM | | COSM4745907 | chr11 | 108203577 | CTTATA | C | medium_deletion |
| ATM | | COSM6853895 | chr11 | 108204619 | TA | T | short_deletion |
| ATM | | COSM6986871 | chr11 | 108205719 | AAATCTGGTG | A | long_deletion |
| | | | | | ACTATAC | | |
| ATM | | COSM200673 | chr11 | 108206581 | G | A | single_base_substitution |
| ATM | | ATM_exon_57_ | chr11 | 108213996 | AACTGTCCCC | A | long_deletion |
| | | indel | | | ATTGGTG | | |
| ATM | | COSM6854263 | chr11 | 108216494 | G | GT | short_insertion |
| ATM | | COSM22484 | chr11 | 108218033 | GACA | G | medium_deletion |
| ATM | | COSM6933059 | chr11 | 108224530 | TGAGACAGTT | T | long_deletion |
| | | | | | CCTTTTA | | |
| ATM | | COSM6930780 | chr11 | 108225581 | ACT | A | medium_deletion |
| ATM | | COSM3733420 | chr11 | 108236089 | GTCT | G | medium_deletion |
| KRAS | K117N | COSM19940 | chr12 | 25378647 | T | G | single_base_substitution |
| KRAS | Q61H | COSM554 | chr12 | 25380275 | T | G | single_base_substitution |
| KRAS | G13D | COSM532 | chr12 | 25398281 | C | T | single_base_substitution |
| CDK4 | R24C | COSM1677139 | chr12 | 58145431 | G | A | single_base_substitution |
| PTPN11 | E76K | COSM13000 | chr12 | 112888210 | G | A | single_base_substitution |
| PTPN11 | G503R | COSM14259 | chr12 | 112926887 | G | A | single_base_substitution |
| BRCA2 | N1784Tfs*7 | COSM18607 | chr13 | 32913836 | CA | C | short_deletion |
| BRCA2 | T3033Lfs*29 | COSM1366491 | chr13 | 32954022 | CA | C | short_deletion |
| AKT1 | E17K | COSM33765 | chr14 | 105246551 | C | T | single_base_substitution |
| MAP2K1 | K57N | COSM1235478 | chr15 | 66727455 | G | T | single_base_substitution |
| IDH2 | R140Q | COSM41590 | chr15 | 90631934 | C | T | single_base_substitution |
| CDH1 | Q23* | COSM19503 | chr16 | 68772218 | C | T | single_base_substitution |
| CDH1 | A634V | COSM19822 | chr16 | 168856093 | C | T | single_base_substitution |
| CDH1 | R732Q | COSM972800 | chr16 | 68862107 | G | A | single_base_substitution |
| TP53 | R342Efs*3 | COSM18597 | chr17 | 7574002 | CG | C | short_deletion |
| TP53 | R273H | COSM10660 | chr17 | 7577120 | C | T | single_base_substitution |
| TP53 | G245C | COSM11081 | chr17 | 7577548 | C | A | single_base_substitution |
| TP53 | R175H | COSM10648 | chr17 | 7578406 | C | T | single_base_substitution |
| TP53 | L26Pfs*11 | COSM45386 | chr17 | 7579700 | CAGAACGTTG | C | long_deletion |
| | | | | | TTTTCAGGAA | | |
| | | | | | GT | | |
| NF1 | I679Dfs*21 | COSM24504 | chr17 | 29553477 | A | AC | short_insertion |
| NF1 | F1247Ifs*18 | COSM436320 | chr17 | 29562656 | CTGTT | C | medium_deletion |
| NF1 | Y2285Tfs*5 | COSM39161 | chr17 | 29665751 | CACTT | C | medium_deletion |
| CDK12 | W719* | COSM118018 | chr17 | 37649051 | G | A | single_base_substitution |
| CDK12 | E928fs27* | COSM6965693 | chr17 | 37671998 | A | AATACACA | long_insertion |
| | | | | | | AAGAT | |
| ERBB2 | L755S | COSM14060 | chr17 | 37880220 | T | C | single_base_substitution |
| ERBB2 | A775_ | COSM20959 | chr17 | 37880981 | A | AGCATACGT | long_insertion |
| | G776insYVMA | | | | | GATG | |
| ERBB2 | V842I | COSM14065 | chr17 | 37881332 | G | A | single_base_substitution |
| BRCA1 | R1443* | COSM979730 | chr17 | 41234451 | G | A | single_base_substitution |
| BRCA1 | K654Sfs*47 | COSM219054 | chr17 | 41245586 | CT | C | short_deletion |
| BRCA1 | E23Vfs*17 | COSM35893 | chr17 | 41276044 | ACT | A | medium_deletion |
| TP53 | | COSM6503572 | chr17 | 7572973 | C | CT | short_insertion |
| TP53 | | COSM9312241 | chr17 | 7576558 | G | A | single_base_substitution |
| ERBB2 | | COSM7345564 | chr17 | 37866636 | GC | G | short_deletion |
| ERBB2 | | COSM94225 | chr17 | 37868208 | C | A | single_base_substitution |
| ERBB2 | | COSM6961097 | chr17 | 37868654 | CAAG | C | medium_deletion |
| ERBB2 | | COSM9110847 | chr17 | 37871547 | C | A | single_base_substitution |
| ERBB2 | | COSM9494227 | chr17 | 37872131 | GCT | G | medium_deletion |
| ERBB2 | | COSM7343981 | chr17 | 37872684 | G | T | single_base_substitution |
| ERBB2 | | COSM6974323 | chr17 | 37873687 | GATGAGGAGG | G | long_deletion |
| | | | | | GCGCATGCCA | | |
| | | | | | GCCTTGCCCC | | |
| ERBB2 | | COSM7345566 | chr17 | 37876051 | TG | T | short_deletion |
| ERBB2 | | COSM6973838 | chr17 | 37879795 | TGGA | T | medium_deletion |
| ERBB2 | | COSM7345570 | chr17 | 37882019 | CG | C | short_deletion |
| SMAD4 | R361H | COSM14122 | chr18 | 48591919 | G | A | single_base_substitution |
| GNA11 | | COSM9232870 | chr19 | 3114987 | CA | C | short_deletion |
| GNA11 | | COSM1392334 | chr19 | 3118933 | TG | T | short_deletion |
| GNAS | R201C | COSM27887 | chr20 | 57484420 | C | T | single_base_ |
| | | | | | | | substitution |
| ARAF | S214C | COSM5044705 | chrX | 47426121 | C | G | single_base_ |
| | | | | | | | substitution |
| AR | W742C | COSM5944171 | chrX | 66937372 | G | C | single_base_ |
| | | | | | | | substitution |
| AR | T878A | COSM236693 | chrX | 66943552 | A | G | single_base_ |
| | | | | | | | substitution |

### List of Fusions Included in the 3rd Party Synthetic Reference Material (Twist) Tested:

| | Left | Left gene | Right | Right gene |
|---|---|---|---|---|
| Gene names | chr:position | (strand) | chr:position | (strand) |
| TRP-ALK | 1:186325170 | TPR (−) | 2:29446942 | ALK (−) |
| NCOA4-RET | 10:51584453 | NCOA4 (+) | 10:43611517 | RET (+) |
| EML4-ALK | 2:42523823 | EML4 (+) | 2:29446684 | ALK (−) |
| EML4-ALK | 2:42501178 | EML4 (+) | 2:29448229 | ALK (−) |
| EML4-ALK | 2:42526356 | EML4 (+) | 2:29447836 | ALK (−) |
| KIF5B-RET | 10:32313599 | KIF5B (−) | 10:43610575 | RET (+) |
| KIF5B-RET | 10:32306826 | KIF5B (−) | 10:43612018 | RET (+) |
| CCDC6-RET | 10:61665683 | CCDC6 (−) | 10:43611678 | RET (+) |
| CCDC6-RET | 10:61616250 | CCDC6 (−) | 10:43611186 | RET (+) |
| CCDC6-RET | 10:61638613 | CCDC6 (−) | 10:43609946 | RET (+) |

### APPENDIX C: REPORTABLE MUTATIONS DETECTED IN 58 LOB SAMPLES

| Sample ID | Mutation Description | VAF | Variant Category |
|---|---|---|---|
| 1382428V2875-2 | BRCA2 p.Q3050* | 0.15% | SNV |
| 1035000B7943-4 | CDH1 p.V391fs*7 | 0.06% | Deletion |

### APPENDIX D: MUTATIONS IN HORIZON SAMPLE DATA

### List of Actionable Mutations Assessed in 3rd Party Reference Standards

| Gene | Mutation Description | hg19 Coordinates | Variant Category |
|---|---|---|---|
| EGFR | L858R | 7:55259515 | SNV |
| EGFR | E746-A750 | 7:55242464 | Deletion |
| EGFR | T790M | 7:55249071 | SNV |
| EGFR | V769_D770insASV | 7:55248998 | Insertion |
| KRAS | G12D | 12:25398284 | SNV |
| NRAS | Q61K | 1:115256530 | SNV |
| NRAS | A59T | 1:115256536 | SNV |
| PIK3CA | E545K | 3:178936091 | SNV |

### APPENDIX E

### Appendix E: Z-Scores from Qc Metrics with HCQCT for Samples Used in Validation (Only with Samples Containing HCQCTS)

| | | Z-Scores from | | | | | | |
|---|---|---|---|---|---|---|---|---|
| Validation | | hcQCT QC Metrics | | | Mean | Sequencing | | Pass/ |
| Study | Sample | EXT | LP | TE | GE | Reads | OTR | Fail |
| Precision | 1377558W1228-4 | 1.04 | −0.07 | 1.18 | 1215 | 8.00E+07 | 85% | Pass |
| | Operator 1 | | | | | | | |
| | 1374173L1834-2 | 1.29 | −0.27 | 0.68 | 2314 | 1.04E+08 | 84% | Pass |
| | Operator1 | | | | | | | |
| | 137525612149-4 | 1.05 | −0.29 | 0.64 | 1966 | 9.96E+07 | 86% | Pass |
| | Operator1 | | | | | | | |
| | 1364321Q2443-2 | 0.16 | −0.41 | 0.34 | 2816 | 1.04E+08 | 85% | Pass |
| | Operator 1 | | | | | | | |
| | 1377055R1435-3 | 1.00 | −0.04 | 0.94 | 1587 | 8.33E+07 | 84% | Pass |
| | Operator1 | | | | | | | |
| | 1378272D1612-4 | 1.11 | −0.10 | 0.54 | 2507 | 1.20E+08 | 84% | Pass |
| | Operator1 | | | | | | | |
| | 1377055D1354-3 | 0.32 | −0.31 | 0.48 | 3350 | 1.28E+08 | 85% | Pass |
| | Operator 1 | | | | | | | |
| | Positive_Control_1 | | 1.08 | 0.50 | 3666 | 1.27E+08 | 85% | Pass |
| | Operator 1 | | | | | | | |
| | Positive_Control_2 | | 0.76 | 0.43 | 3848 | 1.42E+08 | 85% | Pass |
| | Operator1 | | | | | | | |
| | 19-0316_MSI | | 0.97 | 0.77 | 2431 | 1.12E+08 | 85% | Pass |
| | Operator1 | | | | | | | |
| | 14-565_MSI | | 0.89 | 1.42 | 2083 | 1.02E+08 | 85% | Pass |
| | Operator1 | | | | | | | |
| | 21-0189_MSI | | −9.65 | 0.92 | 64 | 6.40E+07 | 85% | QC Fail |
| | Operator 1 | | | | | | | at LP |
| | 21-0189_MSI | | −8.98 | 0.46 | 133 | 6.55E+07 | 89% | QC Fail |
| | Operator1 Redo | | | | | | | at LP |
| | 21-0189_MSI | | −0.95 | 0.52 | 2064 | 1.22E+08 | 88% | Pass |
| | Operator1 Redo | | | | | | | |
| | 1377558W1228-2 | 1.12 | −0.11 | 1.06 | 1166 | 1.11E+08 | 89% | Pass |
| | Operator2 | | | | | | | |
| | 1374173L1834-3 | 1.18 | −0.22 | 1.14 | 2366 | 1.34E+08 | 88% | Pass |
| | Operator2 | | | | | | | |
| | 137525612149-3 | 1.13 | 0.03 | 1.34 | 2285 | 1.20E+08 | 89% | Pass |
| | Operator2 | | | | | | | |
| | 1364321Q2443-4 | 0.43 | −0.22 | 1.47 | 3675 | 1.45E+08 | 89% | Pass |
| | Operator2 | | | | | | | |
| | 1377055R1435-2 | 0.78 | −0.59 | 1.46 | 1393 | 8.53E+07 | 89% | Pass |
| | Operator2 | | | | | | | |
| | 1378272D1612-2 | 0.98 | −1.09 | 1.31 | 2563 | 1.31E+08 | 88% | Pass |
| | Operator2 | | | | | | | |
| | 1377055D1354-2 | 0.49 | −0.83 | 1.21 | 4155 | 1.35E+08 | 89% | Pass |
| | Operator2 | | | | | | | |
| | Positive_Control_1 | | 0.55 | 0.97 | 2916 | 8.09E+07 | 89% | Pass |
| | Operator2 | | | | | | | |
| | Positive_Control_2 | | 0.58 | 0.67 | 3077 | 8.55E+07 | 89% | Pass |
| | Operator2 | | | | | | | |
| | 19-0316_MSI | | 0.31 | 1.12 | 2088 | 7.63E+07 | 89% | Pass |
| | Operator2 | | | | | | | |
| | 14-565_MSI | | 0.38 | 1.73 | 2012 | 8.67E+07 | 90% | Pass |
| | Operator2 | | | | | | | |
| | 21-0189_MSI | | 0.25 | 1.29 | 1966 | 8.03E+07 | 89% | Pass |
| | Operator2 | | | | | | | |

### APPENDIX F: LIST OF GENES COVERED IN NORTHSTAR SELECT ASSAY

| | | | | | | | | | | | |
|---|---|---|---|---|---|---|---|---|---|---|---|
| MTOR | ARID1A* | NRAS* | RIT1 | NTRK1\hat{ } | DDR2 | GATA3 | RET\hat{ }′ | PTEN*′ | FGFR2*\hat{ }′ | HRAS* | CCND1* |
| ATM*′ | CCND2* | KRAS*′ | CDK4*′ | BRCA2*′ | RB1* | AKT1 | MAP2K1 | IDH2 | PALB2* | CDH1* | FANCA |
| TP53* | NF1* | RAD51D* | CDK12* | ERBB2 | BRCA1*′ | RAD51C* | BRIP1* | SMAD4 | STK11 | GNA11 | MAP2K2 |
| JAK3 | CCNE1*′ | ALK\hat{ } | MSH2* | MSH6* | IDH1 | CHEK2* | VHL* | RAF1*′ | MLH1* | CTNNB1 | RHOA |
| PIK3CA*′ | FGFR3\hat{ } | PDGFRA′ | KIT*′ | FBXW7 | TERT | APC* | NPM1 | ROS1\hat{ } | ESR1′ | PMS2* | EGFR*′ |
| CDK6*′ | MET*′ | SMO | BRAF*\hat{ }′ | EZH2 | FGFR1′ | MYC*′ | JAK2 | GNAQ | TSC1 | NOTCH1 | ARAF |
| AR*′ | CD274′ | NTRK3\hat{ }\hat{ } | GNAS | CDKN2A*′ | CDKN2B* | MPL | PTPN11 | AKT2 | FGFR4 | SF3B1 | NTRK2\hat{ }\hat{ } |

*full coding sequence 40 genes
\hat{ }fusion 8 genes
\hat{ }\hat{ }fusion only 2 genes
′also for CNV 24 genes

### ADDITIONAL CONSIDERATIONS

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate+/−10% unless another meaning is apparent from the context. From example, “approximately ten” should be understood to mean “in a range from nine to eleven.”

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs. Thus, while particular embodiments and applications have been illustrated and described, it is to be understood that the described subject matter is not limited to the precise construction and components disclosed. The scope of protection should be limited only by the following claims.

1. A method for genomic profiling of plasma-derived circulating tumor DNA (ctDNA), the method comprising:
obtaining a mixture including plasma from a blood sample of a subject, the mixture including cell-free DNA (cfDNA) from the subject;
performing processing steps on the mixture to produce a sequencing library;
sequencing the sequencing library to produce sequence reads; and
constructing a tumor mutational profile for the subject based on the sequence reads, constructing the tumor mutation profile comprising performing copy number variant (CNV) calling with normalization and auto-exclusion based on an aneuploidy signal.

2. The method of claim 1, wherein the tumor mutational profile further includes an indication of presence or absence of at least of single nucleotide variants (SNVs), insertions and deletions (indels), fusions, or microsatellite instability (MSI).

3. The method of claim 1, wherein the tumor mutational profile further includes an indication of presence of a tumor mutation having a variant allelic fraction (VAF) in a range from 0.05% to 0.2%.

4. The method of claim 1, further comprising:
estimating tumor fraction from cell-free DNA (cfDNA); and
determining an in-tissue CNV for one or more variants identified in the tumor mutation profile.

5. The method of claim 4, wherein estimating the tumor fraction comprises:
applying a Gaussian Mixture Model (GMM) to quantify a liquid aneuploidy signal on measured genes; and
estimating the tumor fraction based on a periodicity pattern determined by the GMM.

6. The method of claim 1, wherein the method uses a panel of multiple genes with a limit of detection of 0.13-0.16% allele fraction for SNVs and indels, and 2.100-2.160 for Copy Number amplification, 1.7-1.9 copies for Copy Number loss, 0.25-0.40% allele fraction for fusions, and 0.07-0.40% allele fraction for MSI.

7. The method of claim 1, wherein at least some of the processing steps including adding a corresponding set of Quantitative Counting Template (QCT) molecules to the mixture, the method further comprising:
performing quality tracking based on a subset of the sequence reads that correspond to the QCT molecules, wherein the tumor mutation profile is constructed responsive to the quality tracking indicating success of the processing steps.

8. The method of claim 7, wherein the processing steps comprise a cfDNA extraction step that extracts cfDNA from the plasma sample, during or before which extraction QCTs are added, wherein a number of sequence reads corresponding to the extraction QCTs indicates success or an issue with the cfDNA extraction step.

9. The method of claim 7, wherein the processing steps comprise a library preparation step that includes at least one of end repair and adaptor ligation, during or before which library preparation QCTs are added, wherein a number of sequence reads corresponding to the library preparation QCTs indicates success of or an issue with the library preparation step.

10. The method of claim 7, wherein the processing steps comprise a selective enrichment step that selectively amplifies target sequences, during or before which target enrichment QCTs are added, wherein a number of sequence reads corresponding to the target enrichment QCTs indicates success of or an issue with the selective enrichment step.

11. The method of claim 7, wherein the quality tracking comprises:
calculating one or more QCT metrics from the sequence reads that correspond to the QCT molecules; and
comparing the one or more QCT metrics to one or more corresponding thresholds, wherein the success of the processing steps is indicated by the one or more QCT metrics meeting requirements relative to the corresponding thresholds.

12. The method of claim 11, wherein the one or more QCT metrics comprise at least one of a z-score, a sequence saturation level, or a base-wise error rate.

13. The method of claim 1, wherein the sequencing has a median probe coverage of at least 40,000×.

14. The method of claim 1, further comprising false-positive filtering and calling of SNVs and Indels.

15. The method of claim 14, wherein the false-positive filtering comprises adaptive filtering.

16. The method of claim 1, further comprising performing copy number noise reduction for a batch comprising a plurality of samples, the plurality of samples including the blood sample, wherein performing the copy number noise reduction comprises:
averaging raw coverages per gene per sample of the plurality of samples, the raw coverages measured at a center of each of a plurality of probes;
normalizing the normalized coverages to a median gene-level coverage;
filtering the normalized coverages to obtain a subset of the plurality of probes;
creating a model for each sample to predict sample-normalized coverage for guanine-cytosine (GC) content of the subset of the plurality of probes;
normalizing the sample-normalized coverages to expected values from the models corresponding to the samples to determine per-sample GC normalized coverage;
removing, for all of the plurality of samples, probe values that deviate from the expected value by at least a threshold amount for at least one of the plurality of samples;
measuring a median per-sample GC normalized coverage for each probe across the batch;
normalizing the per-sample GC normalized coverage to generate expected GC normalized values; and
calculating probe copy numbers from the expected GC normalized values.

17. The method of claim 16, wherein the subset of the plurality of probes comprises those of the plurality of probes that are well-behaved.

18. The method of claim 1, further comprising estimating an in-tissue copy number of a focal CNV from a plasma measurement.

19. The method of claim 1, further comprising:
extracting buffy coat DNA from the blood sample;
analyzing the buffy coat DNA to identify a set of one or more mutations present in the buffy coat DNA; and
filtering mutations included in the tumor mutation profile to not include at least some of the set of one or more mutations.

20. The method of claim 19, wherein analyzing the buffy coat DNA comprises performing a Clonal Hematopoiesis of Indeterminate Potential (CHIP) analysis.

21. The method of claim 19, wherein analyzing the buffy coat DNA comprises using a bespoke digital droplet PCR (ddPCR) operation that is designed using the sequencing reads from the sequencing library.

22. The method of claim 19, wherein analyzing the buffy coat DNA comprises performing Comprehensive Genome Profiling to identify CHIP mutations.

23. The method of claim 19, wherein analyzing the buffy coat DNA comprises performing a bespoke multiplex PCR operation that is designed using the sequencing reads from the sequencing library.

24. The method of claim 1, further comprising performing tumor fraction estimation based on one or more signals derived from plasma of the blood sample, the one or more signals including at least one of: a maximum/average SNV signal, the aneuploidy signal, or a methylation signal.

25. The method of claim 1, wherein the aneuploidy signal is determined by:
obtaining copy number data for the plasma of the blood sample;
fitting a Gaussian Mixture Model (GMM) to the copy number data, the fitting of the GMM generating a plurality of peaks distributed around a central peak; and
inferring a tumor fraction from distances between the plurality of peaks from the central peak.

26. A method of performing copy number noise reduction for a batch comprising a plurality of blood samples, wherein the method comprises:
obtaining sequencing data for the plurality of blood samples;
averaging raw coverages per gene per sample in the sequencing data, the raw coverages measured at a center of each of a plurality of probes;
normalizing the normalized coverages to a median gene-level coverage;
filtering the normalized coverages to obtain a subset of the plurality of probes;
creating a model for each sample to predict sample-normalized coverage for guanine-cytosine (GC) content of the subset of the plurality of probes;
normalizing the sample-normalized coverages to expected values from the models corresponding to the samples to determine per-sample GC normalized coverage;
removing, for all of the plurality of samples, probe values that deviate from the expected value by at least a threshold amount for at least one of the plurality of samples;
measuring a median per-sample GC normalized coverage for each probe across the batch;
normalizing the per-sample GC normalized coverage to generate expected GC normalized values; and
calculating probe copy numbers from the expected GC normalized values.

27. A method for genomic profiling of plasma-derived circulating tumor DNA (ctDNA), the method comprising:
obtaining a mixture including plasma from a blood sample of a subject, the mixture including cell-free DNA (cfDNA) from the subject;
performing processing steps on the mixture to produce a sequencing library, wherein at least some of the processing steps including adding a corresponding set of Quantitative Counting Template (QCT) molecules to the mixture;
sequencing the sequencing library to produce sequence reads;
performing quality tracking based on a subset of the sequence reads that correspond to the QCT molecules; and
responsive to the quality tracking indicating success of the processing steps, constructing a tumor mutational profile for the subject based on the sequence reads.

28. The method of claim 27, further comprising estimating a tumor fraction of the ctDNA, wherein the tumor mutation profile is further based on the tumor fraction.

29. A method of genomic profiling of circulating tumor DNA (ctDNA), the method comprising:
analyzing plasma extracted from a blood sample using a hybrid capture method that interrogates millions of base pairs;
designing a bespoke multiplex PCR or ddPCR operation to evaluate whether a set of mutations identified using the hybrid capture method are present in buffy coat from the blood sample;
extracting buffy coat DNA from the blood sample;
performing the multiplex PCR or ddPCR operation on the buffy coat DNA;
identifying, based on results of the multiplex PCR or ddPCR operation, that one or more of the set of mutations are present in the buffy coat DNA; and
generating a tumor mutation profile that reports mutations detected by the hybrid capture method excluding the one or more of the set of mutations that are present in the buffy coat.

30. The method of claim 29, wherein the set of mutations comprises 1-10 mutations.
